(19) United States
(12) Patent Application Publication
(10) Pub. No.: US 2002/0052007 A1
(43) Pub. Date: May 2, 2002

(54) Diarylmethylbenzylpiperazines and corresponding halobenzyl derivatives

(76) Inventors: Kwen Jen Chang, Chapel Hill, NC (US); William Pendergast, Durham, NC (US); Kestutis P. Biciunas, Durham, NC (US); Edwin Shen-Chou Wu, Cary, NC (US)

Correspondence Address: Steven J. Hultquist, P.O. Box 14329, Research Triangle Park, NC, 27709, US

(21) Appl. No.: 09/957,903

(22) Filed: Sep. 21, 2001

Related U.S. Application Data
(63) Continuation-in-part of application No. 09/352,308, filed on Jul. 12, 1999, now Pat. No. 6,300,332.
Application No. 09/352,308 is a division of application No. 08/887,312, filed on Jul. 3, 1997, now Pat. No. 5,985,880.
Application No. 08/887,312 is a continuation-in-part of application No. 08/658,726, filed on Jun. 5, 1996, now Pat. No. 5,807,858.

(30) Foreign Application Priority Data
Feb. 2, 1993 (GB) PCT/GB93/00216
Feb. 3, 1992 (GB) 9202238.3

(51) Int. Cl. G01N 33/53

(52) U.S. Cl. 435/7.1

(57) ABSTRACT

Diarylmethylbenzylpiperazine compounds, optionally substituted on an aromatic ring of the benzyl substituent with one or more halo substituents, and pharmaceutically acceptable esters and salts of such compounds. Such compounds are usefully employed in therapeutic intervention for treatment or prophylaxis of disease states and conditions such as drug addiction, alcohol addiction, drug overdose, mental illness, cough, lung edema, gastro-intestinal disorders, arthritis, psoriasis, asthma, inflammatory bowel disease, disorders of respiratory function, functional bowel disease, irritable bowel syndrome, diarrhea, functional distension, pain (e.g., functional pain, trauma pain, etc.), non-ulcerogenic dyspepsia, urogenital tract disorders, organ transplant rejection, skin graft rejection, mental disorders, cognitive disorders, emesis, respiratory depression, acne and skin lesions. One preferred compound of such type is

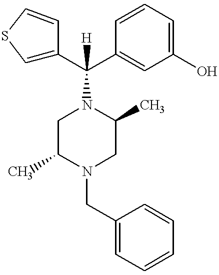

or a pharmaceutically acceptable ester or salt thereof.

### BACKGROUND OF THE INVENTION

1. Field of the Invention

The present invention relates to diarylmethylbenzylpiperazines and corresponding halobenzyl derivatives thereof, to methods of making such diarylmethylbenzylpiperazines and halobenzyl derivatives, and to methods of using such diarylmethylbenzylpiperazines and halobenzyl derivatives for therapeutic intervention.

2. Description of the Related Art

In the study of opioid biochemistry, a variety of endogenous opioid compounds and non-endogenous opioid compounds has been identified. In this effort, significant research has been focused on understanding the mechanism of opioid drug action, particularly as it relates to cellular and differentiated tissue opiate receptors.

Opioid drugs typically are classified by their binding selectivity in respect of the cellular and differentiated tissue receptors to which a specific drug species binds as a ligand. These receptors include mu (μ), delta (δ), sigma (σ) and kappa (κ) receptors.

The well-known narcotic opiates, such as morphine and its analogs, are selective for the opiate mu receptor. Mu receptors mediate analgesia, respiratory depression, and inhibition of gastrointestinal transit. Kappa receptors mediate analgesia and sedation. Sigma receptors mediate various biological activities.

Diarylmethyl piperazine compounds are variously described in Chang et al. U.S. Pat. No. 5,658,908 issued Aug. 19, 1997; U.S. Pat. No. 5,681,830 issued Oct. 28, 1997; U.S. Pat. No. 5,552,404 issued Sep. 3, 1996; U.S. Pat. No. 5,574,159 issued Nov. 12, 1996; U.S. Pat. No. 5,854,249 issued Dec. 29, 1998; U.S. Pat. No. 5,807,858 issued Sep. 15, 1998; and U.S. Pat. No. 5,985,880 issued Nov. 16, 1999, as having delta receptor and mixed mu/delta receptor binding activity and therapeutic utility, e.g., for mediating analgesia, and treatment/prophylaxis of various disease states and physiological conditions including diarrhea, mental illness, drug and alcohol addiction/overdose, lung edema, depression, asthma, emphysema, cough, apnea, respiratory depression, cognitive disorders, emesis and gastrointestinal disorders.

The present invention relates to diarylmethylpiperazine compounds of specific type, and their synthesis and use.

### SUMMARY OF THE INVENTION

The present invention relates in one aspect to diarylmethylpiperazine compounds of the general formula:

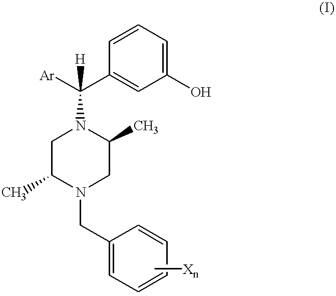

wherein:

Ar is a 5- or 6-member carbocyclic or heterocyclic aromatic ring with atoms selected from the group consisting of carbon, nitrogen, oxygen and sulfur, and having on a first carbon atom thereof a substituent Y and on a second ring carbon thereof a substituent R¹,

Y is selected from the group consisting of:

hydrogen;

halogen;

C₁-C₆ alkyl, C₂-C₆ alkenyl, C₂-C₆ alkynyl;

C₁-C₆ haloalkyl;

C₁-C₆ alkoxy;

C₃-C₆ cycloalkoxy;

sulfides of the formula SR⁸ where R⁸ is C₁-C₆ alkyl, C₂-C₆ alkenyl, C₂-C₆ alkynyl, C₃-C₆ cycloalkyl, arylalkyl having a C₅-C₁₀ aryl moiety and an C₁-C₆ alkyl moiety, or C₅-C₁₀ aryl;

sulfoxides of the formula SOR⁸ where R⁸ is the same as above;

sulfones of the formula SO₂R⁸ where R⁸ is the same as above;

nitrile;

C₁-C₆ acyl;

alkoxycarbonylamino (carbamoyl) of the formula NHCO₂R⁸ where R⁸ is the same as above;

carboxylic acid, or an ester, amide, or salt thereof;

aminomethyl of the formula CH₂NR⁹R¹⁰ where R⁹ and R¹⁰ may be the same or different, and may be hydrogen, C₁-C₆ alkyl, C₂-C₆ alkenyl, C₂-C₆ alkynyl, C₂-C₆ hydroxyalkyl, C₂-C₆ methoxyalkyl, C₃-C₆ cycloalkyl, or C₅-C₁₀ aryl, or R⁹ and R¹⁰ together may form a ring of 5 or 6 atoms, the ring atoms selected from the group consisting of N and C;

carboxamides of the formula CONR⁹R¹⁰ where R⁹ and R¹⁰ are the same as above, or C₂-C₃₀ peptide conjugates thereof; and

sulfonamides of the formula SO₂NR⁹R¹⁰ where R⁹ and R¹⁰ are the same as above;

R¹ is hydrogen, halogen, or C₁-C₄ alkyl, C₂-C₄ alkenyl, C₁-C₄ alkynyl;

X is halo (fluorine, bromine, chlorine, iodine), preferably fluoro; and

n is from 0 to 5;

or a pharmaceutically acceptable ester or salt thereof.

In one particularly preferred aspect, the invention relates to a compound of the formula (II):

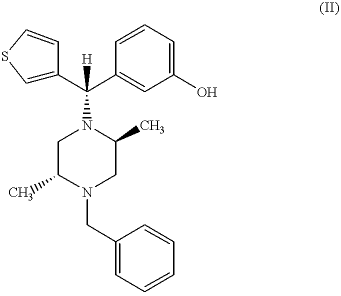

3-((S)-((2S,5R)-4-Benzyl-2,5-dimethyl-1-piperazinyl)(3-thienyl)methyl)phenol, sometimes hereinafter referred to as DPI-1 31, or a pharmaceutically acceptable ester or salt thereof.

In another particularly preferred aspect, the invention relates to a compound of the formula (VIII):

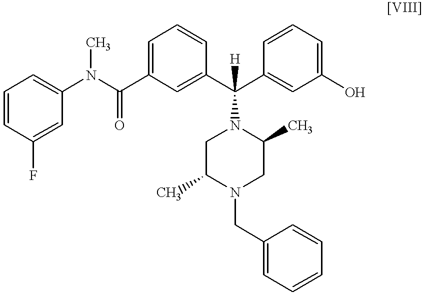

3-((R)-((2S,5R)-2,5-dimethyl-4-(4-fluoro-benzyl)-1-piperazinyl)-3-hydroxybenzyl)-N-(3-fluorophenyl)-N-methylbenzamide, sometimes hereinafter referred to as DPI-130, or a pharmaceutically acceptable ester or salt thereof.

A further aspect of the invention relates to a diarylmethylpiperazine compound substituted on the piperazine ring with a benzyl substituent which in turn is optionally substituted on the phenyl ring of the benzyl group with at least one halogen substituent.

Another aspect of the invention relates to a method of treatment or prophylaxis of a condition selected from the group consisting of drug addiction, alcohol addiction, drug overdose, mental illness, cough, lung edema, gastro-intestinal disorders, arthritis, psoriasis, asthma, inflammatory bowel disease, disorders of respiratory function, functional bowel disease, irritable bowel syndrome, diarrhea, functional distension, pain (e.g., functional pain, trauma pain, etc.), non-ulcerogenic dyspepsia, urogenital tract disorders, organ transplant rejection, skin graft rejection, cardiac disorders, mental disorders, emotional disorders, cognitive disorders; emesis; respiratory depression; acne, skin lesions, etc. The method comprises administering to a subject in need of such treatment or prophylaxis an effective amount therefor of a diarylmethylbenzylpiperazine compound, optionally substituted on an aromatic ring of the benzyl substituent with one or more halo substituents, or a pharmaceutically acceptable ester or salt of such compound.

A still further aspect of the invention relates to a method of treatment or prophylaxis of pain, comprising administering to a subject in need of such treatment or prophylaxis an effective amount therefor of a compound of formula (I), e.g., a compound of formula (II) or formula (VIII), or a pharmaceutically acceptable ester or salt thereof.

An additional aspect of the invention relates to a method of diagnosis of degeneration or dysfunction of delta opioid receptors associated with a disease state or physiological condition involving tissue or discrete cellular loci comprising such receptors, the method comprising administration of a labeled delta opioid receptor-binding compound to a subject to effect binding of the compound, and determining the extent of binding of the compound to the delta opioid receptors in the subject, as diagnostic information for such diagnosis.

Another aspect of the invention relates to a method of treatment or prophylaxis of a disease state or physiological condition, comprising administering to a subject in need thereof, a diarylmethylpiperazine compound having a safety ratio of at least 12.

Various other aspects, features and embodiments of the invention will be more fully apparent from the ensuing disclosure and appended claims.

### DETAILED DESCRIPTION OF THE INVENTION, AND PREFERRED EMBODIMENTS THEREOF

The disclosures of the following U.S. patents and patent applications are hereby incorporated herein by reference, in their respective entireties:

Chang et al. U.S. Pat. No. 5,658,908 issued Aug. 19, 1997;

Chang et al. U.S. Pat. No. 5,681,830 issued Oct. 28, 1997;

Chang et al. U.S. Pat. No. 5,552,404 issued Sep. 3, 1996;

Chang et al. U.S. Pat. No. 5,574,159 issued Nov. 12, 1996;

Chang et al. U.S. Pat. No. 5,854,249 issued Dec. 29, 1998;

Chang et al. U.S. Pat. No. 5,807,858 issued Sep. 15, 1998;

Chang et al. U.S. Pat. No. 5,985,880 issued Nov. 16, 1999; and

Chang et al. U.S. patent application Ser. No. 09/352,308 filed Jul. 12, 1999, now allowed.

The compounds of the present invention broadly include diarylmethylpiperazine compounds of the general formula:

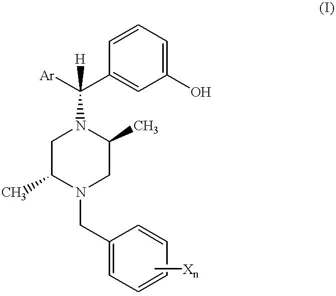

wherein:

Ar is a 5- or 6-member carbocyclic or heterocyclic aromatic ring with atoms selected from the group consisting of carbon, nitrogen, oxygen and sulfur, and having on a first carbon atom thereof a substituent Y and on a second ring carbon thereof a substituent R¹,

Y is selected from the group consisting of:

hydrogen;

halogen;

C₁-C₆ alkyl, C₂-C₆ alkenyl, C₂-C₆ alkynyl;

C₁-C₆ haloalkyl;

C₁-C₆ alkoxy;

C₃-C₆ cycloalkoxy;

sulfides of the formula SR⁸ where R⁸ is C₁-C₆ alkyl, C₂-C₆ alkenyl, C₂-C₆ alkynyl, C₃-C₆ cycloalkyl, arylalkyl having a C₅-C₁₀ aryl moiety and an C₁-C₆ alkyl moiety, or C₅-C₁₀ aryl;

sulfoxides of the formula SOR⁸ where R⁸ is the same as above;

sulfones of the formula SO₂R⁸ where R⁸ is the same as above;

nitrile;

C₁-C₆ acyl;

alkoxycarbonylamino (carbamoyl) of the formula NHCO₂R⁸ where R⁸ is the same as above;

carboxylic acid, or an ester, amide, or salt thereof;

aminomethyl of the formula CH₂NR⁹R¹⁰ where R⁹ and R¹⁰ may be the same or different, and may be hydrogen, C₁-C₆ alkyl, C₂-C₆ alkenyl, C₂-C₆ alkynyl, C₂-C₆ hydroxyalkyl, C₂-C₆ methoxyalkyl, C₃-C₆ cycloalkyl, or C₅-C₁₀ aryl, or R⁹ and R¹⁰ together may form a ring of 5 or 6 atoms, the ring atoms selected from the group consisting of N and C; carboxamides of the formula CONR⁹R¹⁰ where R⁹ and R¹⁰ are the same as above, or C₂-C₃₀ peptide conjugates thereof; and sulfonamides of the formula SO₂NR⁹R¹⁰ where R⁹ and R¹⁰ are the same as above;

R¹ is hydrogen, halogen, or C₁-C₄ alkyl, C₂-C₄ alkenyl, C₁-C₄ alkynyl;

X is halo (fluorine, bromine, chlorine, iodine), preferably fluoro; and

n is from 0 to 5;

or a pharmaceutically acceptable ester or salt thereof.

Ar in the above formula may be thiophenyl, thiazolyl, furanyl, pyrrolyl, phenyl, or pyridyl, being preferably phenyl or thiophenyl, and most preferably thiophenyl. Illlustrative compounds of the invention include those of formulae (II)-(XV):

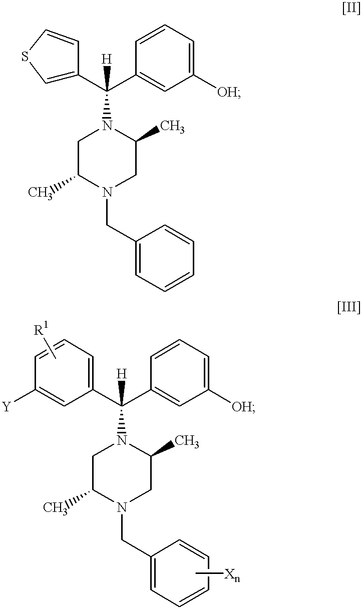

wherein Y, R¹, n and x are as defined in formula (I) above;

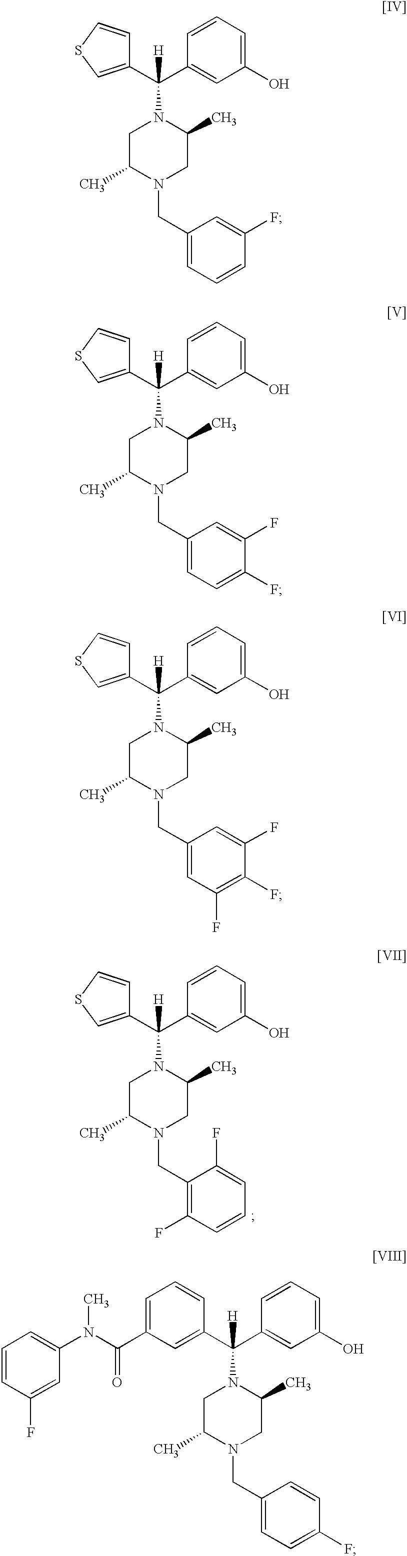

as well as their pharmaceutically acceptable esters and salts.

Particularly preferred compounds of the invention include those of formulae (II) and (VIII), as well as their pharmaceutically acceptable esters and salts.

While the compounds of the invention are described hereinafter with primary reference to diarylmethylbenzylpiperazines and halobenzyl derivatives thereof, including their respective ester and salt forms, it will be recognized that the methods of the invention for treatment or prophylaxis of various disease states and physiological conditions may include use of a wide variety of diarylmethylpiperazines wherein the piperazinyl ring has an arylalkyl substituent, e.g., arylalkyl having C₁-C₁₀ aryl and C₁-C₆ alkyl moieties.

The compounds of the invention have utility in treatment or prophylaxis in a variety of indications, including, without limitation, drug addiction, alcohol addiction, drug overdose, mental illness, cough, lung edema, sexual dysfunction, gastro-intestinal disorders, arthritis, psoriasis, asthma, inflammatory bowel disease, disorders of respiratory function, functional bowel disease, irritable bowel syndrome, diarrhea, functional distension, pain (e.g., functional pain, trauma pain, etc.), non-ulcerogenic dyspepsia, urogenital tract disorders, organ transplant rejection, skin graft rejection, cardiac disorders, mental disorders, emotional disorders, cognitive disorders; emesis; respiratory depression; acne and skin lesions.

In a broad method aspect of the present invention, a diarylmethylbenzylpiperazine compound, optionally substituted on an aromatic ring of said benzyl substituent with one or more halo substituents, or a pharmaceutically acceptable ester or salt of such compound, is administered to a subject in need of treatment or prophylaxis of a condition specified in the preceding paragraph, for such treatment or prophylaxis. Such compound may be a compound of the aforementioned formula (I), including any compounds of the various formulae (II)-(XV) described hereinabove.

In a particularly preferred method of the invention, treatment or prophylaxis of pain is effected by administering to a subject in need of such treatment or prophylaxis an effective amount of a compound of formula (11) or a pharmaceutically acceptable ester or salt thereof.

Examples of pharmaceutically acceptable esters of the compound of formula (I) include carboxylic acid esters of the hydroxyl group in the compound of formula (I) in which the non-carbonyl moiety of the carboxylic acid portion of the ester grouping is selected from straight or branched chain alkyl (e.g. n-propel, t-butyl, n-butyl), alkoxyalkyl (e.g. methoxymethyl), arylalkyl (e.g. benzyl), aryloxyalky (e.g. phenoxymethyl), and aryl (e.g. phenyl); alkyl-, aryl-, or arylalkylsulfonyl (e.g. methanesulfonyl); amino acid esters (e.g. L-valyl or L-isoleucyl); dicarboxylic acid esters (e.g. hemisuccinate); carbonate esters (e.g. ethoxycarbonyl); carbamate esters (e.g. dimethylaminocarbonyl, (2-aminoethyl)aminocarbonyl); and inorganic esters (e.g. mono-, di- or triphosphate).

Examples of pharmaceutically acceptable salts of the compound of formula (I) include salts derived from an appropriate base, such as an alkali metal (for example, sodium, potassium), an alkaline earth metal (for example, calcium, magnesium), ammonium and NR′₄⁺ (wherein R′ is C₁-C₄ alkyl). Pharmaceutically acceptable salts of an amino group include salts of: organic carboxylic acids such as acetic, lactic, tartaric, malic, lactobionic, fumaric, and succinic acids; organic sulfonic acids such as methanesulfonic, ethanesulfonic, isethionic, benzenesulfonic and p-toluenesulfonic acids; and inorganic acids such as hydrochloric, hydrobromic, sulfuric, phosphoric and sulfamic acids. Pharmaceutically acceptable salts of a compound having a hydroxyl group consist of the anion of said compound in combination with a suitable cation such as Na⁺, NH₄⁺, or NR′₄⁺ (wherein R′ is for example a C₁-C₄ alkyl group).

For therapeutic use, salts of the compound of formula (I) will be pharmaceutically acceptable, i.e., they will be salts derived from a pharmaceutically acceptable acid or base. However, salts of acids or bases that are not pharmaceutically acceptable may also find use, for example, in the preparation or purification of a pharmaceutically acceptable compound. All salts, whether or not derived from a pharmaceutically acceptable acid or base, are within the scope of the present invention.

The compounds of formula (I) have utility as exogenous receptor combinent compounds, and may be used for binding with an opioid receptor. Further, the compounds may be used as a conjugate in an agonist/antagonist pair that is employed for transductional assay of neurotransmitter function in appertaining cellular or differentiated tissue systems, as well as for receptor assay, differential binding, and specificity applications for cellular, histological, and corporeal monitoring and assessment purposes.

The compounds of the above formula (I) exhibit specific bioactivity characteristics rendering them useful as therapeutic agents for treatment or prophylaxis of a wide variety of physiological and pathological conditions.

The compounds of formula (I) are particularly useful in mediating analgesia with reduced respiratory depression, as well as for the treatment of various disease states and physiological conditions, including, without limitation, diarrhea, mental illness, apnea, cognitive disorders, cardiac disorders, cough, lung edema, gastrointestinal disorders, spinal injury, and drug addiction.

The compounds of formula (I) also are usefully employed in combating respiratory, muscular or nausea side effects of mu receptor agonist therapeutic agents when administered concurrently or contemporaneously with such mu receptor agonist therapeutic agents. The present invention therefore contemplates a pharmaceutical composition comprising (i) a mu receptor agonist therapeutic agent mediating respiratory, muscular and/or nausea side effects and (ii) an effective amount for reducing, treating or preventing the side effects, of a compound of formula (I).

In such combination of the side effect-mediating therapeutic agent and the compound of formula (I), the dosage of the therapeutic agent, and the dosage of the formula (I) compound for reducing, treating or preventing the side effect, can be independently determined. The separate control of dosages for these two functions provides for greater flexibility in treating individual patients.

In addition to methods of therapeutic intervention, the present invention also provides methods for screening and characterizing respiratory depression-suppressing compounds, comprising conducting activity reversal assays of candidate respiratory depression-suppressing compounds which in receptor tissue transductionally mediate a respiratory depression-supressing effect in response to a respiration-depressing composition.

The activity reversal assays are conducted comparatively, in the absence and in the presence of a compound of formula (I), to determine if the (respiratory depression) suppressing activity of the candidate compound is markedly reversed in the receptor system by the presence of the compound of formula (I). If so, the assay indicates the candidate respiratory depression-suppressing compound as possessing potential bioefficacy for suppressing respiratory depression effects incident to the use of other therapeutic agents.

The compounds of formula (I) can be administered for therapeutic intervention in a pharmaceutical composition containing the compound and a pharmaceutically acceptable carrier. The invention contemplates the use of any means and/or of modality of administration of the compositions of the invention.

Compounds of the above general formula (I) exhibit binding selectivity for receptor(s). Depending on the structure and stereo-specificity of the particular formula (I) compounds, such compounds may exhibit binding ability to receptor(s) selected from the group consisting of delta receptors, mu receptors, kappa receptors, sigma receptors, and combinations of such receptors.

Various compounds within general formula (I) exhibit delta receptor agonist activity. In the case of delta receptor agonists, activity is generally distinguished and measured by activity in the electrically stimulated mouse vas deferens assay, as well as in mouse brain assay involving the existence of a delta receptor subtype that is different from the delta receptor in the mouse vas deferens.

In consequence of the existence of delta receptor subtypes, other receptor binding assays or screening techniques may be employed as a further predictor of agonist or antagonist activity for specific compounds of the present invention.

In addition, to the extent that degeneration or dysfunction of opioid receptors is present or implicated in a disease state involving tissue or discrete cellular loci, isotopically labeled versions of the opioid compounds of the present invention may find utility in diagnostic and imaging applications, e.g., diagnostic techniques involving positron emission tomography (PET) scans of the brain.

For example, a method of diagnosis of degeneration or dysfunction of delta opioid receptors associated with a disease state or physiological condition involving tissue or discrete cellular loci comprising such receptors, may be carried out by administration of a labeled delta opioid receptor-binding compound to a subject to effect binding of the compound to the delta opioid receptors in the subject, followed by determination of the extent of binding of the compound to the delta opioid receptors in the subject, as diagnostic information for the diagnosis. The delta opioid receptor-binding compound may for example be labeled by fluorescent, isotopic or reporter group labeling. In one preferred aspect, the extent of binding of the compound to the delta opioid receptors in the subject, is determined using positron emission tomography.

The delta opioid receptor binding compound may be of any suitable type, e.g., a diarylmethylpiperazine compound optionally substituted on the piperazine ring with a benzyl substituent which in turn is optionally substituted on the phenyl ring of the benzyl group with at least one halogen substituent. In one preferred aspect, such compound is a diarylmethylbenzylpiperazine compound of formula (I) or a pharmaceutically acceptable ester or salt thereof.

As used herein, in reference to the present invention, the term “alkyl” is intended to be broadly construed as encompassing: (i) alkyl groups of straight-chain as well as branched chain character; (ii) unsubstituted as well as substituted alkyl groups, wherein the substituents of substituted alkyl groups may include any sterically acceptable substituents which are compatible with such alkyl groups and which do not preclude the efficacy of the diarylmethylbenzylpiperazine compound for its intended utility (examples of substituents for substituted alkyl groups include halogen (e.g., fluoro, chloro, bromo, and iodo), amino, amido, C₁-C₄ alkyl, C₁-C₄ alkoxy, nitro, hydroxy, etc.); (iii) saturated alkyl groups as well as unsaturated alkyl groups, the latter including groups such as alkenyl-substituted alkyl groups (e.g., allyl, methallyl, propallyl, butenylmethyl, etc.), alkynyl-substituted alkyl groups, and any other alkyl groups containing sterically acceptable unsaturation which is compatible with such alkyl groups and which does not preclude the efficacy of the diarylmethylbenzylpiperazine compound for its intended utility; and (iv) alkyl groups including linking or bridge moieties, e.g., heteroatoms such as nitrogen, oxygen, sulfur, etc.

As used herein, in reference to the present invention, the term “aryl” is intended to be broadly construed as referring to carbocyclic (e.g., phenyl, naphthyl) as well as heterocyclic aromatic groups (e.g., pyridyl, thienyl, furanyl, etc.) and encompassing unsubstituted as well as substituted aryl groups, wherein the substituents of substituted aryl groups may include any sterically acceptable substituents which are compatible with such aryl groups and which do not preclude the efficacy of the diarylmethylbenzylpiperazine compound for its intended utility. Examples of substituents for substituted aryl groups include one or more of halogen (e.g., fluoro, chloro, bromo, and iodo), amino, amido, C₁-C₄ alkyl, C₁-C₄ alkoxy, nitro, trifluoromethyl, hydroxy, hydroxyalkyl containing a C₁-C₄ alkyl moiety, etc.

The compounds contemplated by the invention include those of formula (I) per se, as well as physiologically functional derivatives thereof.

By “physiologically functional derivative” is meant a pharmaceutically acceptable salt, ether, ester or salt of an ether or ester of the compound of formula (I) or any other compound which, upon administration to the recipient, is capable of providing (directly or indirectly) the said compound of formula (I) or an active metabolite or residue thereof. Phenolic C₁-C₆ alkyl ethers are a sub-class of physiologically functional derivatives of the compounds of formula (I).

In enantiomeric forms, compounds of the invention include individual enantiomers of the compounds of formula (I) in single species form substantially free of the corresponding enantiomer, as well as in admixture (in mixtures of enantiomeric pairs and/or in mixtures of multiple enantiomer species).

As used herein, the term “hydrocarbyl” is intended to encompass a group containing only carbon and hydrogen atoms, which may contain double or triple bonds and which may be cyclic or aromatic in nature.

The compounds of the present invention may be readily synthesized within the skill of the art and in view of the illustrative synthetic examples hereinafter set forth.

The compounds of the invention when used in pharmaceutical or diagnostic applications desirably are prepared in substantially pure enantiomer form, with an enantiopurity of at least 90% enantiomeric excess (EE), preferably at least 95% EE, more preferably at least 98% EE, and most preferably at least 99% EE. Enantiomeric excess values provide a quantitative measure of the excess of the percentage amount of a major isomer over the percentage amount of a minor isomer which is present therewith, and may be readily determined by suitable methods well-known and established in the art, as for example chiral high pressure liquid chromatography (HPLC), chiral gas chromatography (GC), nuclear magnetic resonance (NMR) using chiral shift reagents, etc.

Subjects to be treated by the methods of the present invention include both human and non-human animal (e.g., bird, dog, cat, cow, horse) subjects, and are preferably mammalian subjects, and most preferably human subjects. Depending on the specific condition to be treated, animal subjects may be administered compounds of formula (I) at any suitable therapeutically effective and safe dosage, as may readily be determined within the skill of the art, and without undue experimentation.

In in vitro tests for agonist/antagonist activity, such as receptor binding affinity tests, and inhibition of electrically stimulated muscle twitch tests, compounds of the present invention exhibit potency over a range of from nanomolar to micromolar concentrations, depending on the specific compound employed.

In general, while the effective dosage of compounds of the invention for therapeutic use may be widely varied in the broad practice of the invention, depending on the specific application, condition, or disease state involved, as readily determinable within the skill of the art, suitable therapeutic doses of the compounds of the invention, for each of the appertaining compositions described herein, and for achievement of therapeutic benefit in treatment of each of the conditions described herein, will be in the range of 10 micrograms (μg) to 100 milligrams (mg) per kilogram body weight of the recipient per day, preferably in the range of 50 μg to 75 mg per kilogram body weight per day, and most preferably in the range of 100 μg to 50 mg per kilogram body weight per day. The desired dose is preferably presented as two, three, four, five, six, or more sub-doses administered at appropriate intervals throughout the day. These sub-doses may be administered in unit dosage forms, for example, containing from 10 μg to 1000 mg, preferably from 50 μg to 500 mg, more preferably from 50 μg to 250 mg, and most preferably from 50 μg to 10 mg of active ingredient per unit dosage form. Alternatively, if the condition of the recipient so requires, the doses may be administered as a continuous infusion.

The mode of administration and dosage forms will of course affect the therapeutic amounts of the compounds which are desirable and efficacious for the given treatment application.

For example, orally administered dosages typically are at least twice, e.g., 2-10 times, the dosage levels used in parenteral administration methods, for the same active ingredient. In oral administration, dosage levels for delta receptor binding compounds of the invention may be on the order of 5-200 mg/70 kg body weight/day. In tablet dosage forms, typical active agent dose levels are on the order of 10-100 mg per tablet.

The compounds of formula (I) may be administered per se as well as in the form of pharmaceutically acceptable esters, salts, and ethers, as well as other physiologically functional derivatives of such compounds.

The present invention also contemplates pharmaceutical formulations, both for veterinary and for human medical use, which comprise as the active agent one or more compound(s) of the invention.

In such pharmaceutical formulations, the active agent preferably is utilized together with one or more pharmaceutically acceptable carrier(s) therefor and optionally any other therapeutic ingredients. The carrier(s) must be pharmaceutically acceptable in the sense of being compatible with the other ingredients of the formulation and not unduly deleterious to the recipient thereof. The active agent is provided in an amount effective to achieve the desired pharmacological effect, as described above, and in a quantity appropriate to achieve the desired daily dose.

The formulations include those suitable for parenteral as well as non-parenteral administration, and specific administration modalities include oral, rectal, topical, sub-lingual, mucosal, nasal, ophthalmic, subcutaneous, intramuscular, intravenous, transdermal, spinal, intrathecal, intra-articular, intra-arterial, sub-arachnoid, bronchial, lymphatic, and intra-uterine administration. Formulations suitable for parenteral administration are preferred.

When the active agent is utilized in a formulation comprising a liquid solution, the formulation advantageously may be administered parenterally. When the active agent is employed in a liquid suspension formulation or as a powder in a biocompatible carrier formulation, the formulation may be advantageously administered orally, rectally, or bronchially.

When the active agent is utilized directly in the form of a powdered solid, the active agent may advantageously administered orally. Alternatively, it may be administered bronchially, via nebulization of the powder in a carrier gas, to form a gaseous dispersion of the powder which is inspired by the patient from a breathing circuit comprising a suitable nebulizer device.

In some applications, it may be advantageous to utilize the active agent in a “vectorized” form, such as by encapsulation of the active agent in a liposome or other encapsulant medium, or by fixation of the active agent, e.g., by covalent bonding, chelation, or associative coordination, on a suitable biomolecule, such as those selected from proteins, lipoproteins, glycoproteins, and polysaccharides.

The formulations comprising the active agent of the present invention may conveniently be presented in unit dosage forms and may be prepared by any of the methods well known in the art of pharmacy. Such methods generally include the step of bringing the active compound(s) into association with a carrier that constitutes one or more accessory ingredients. Typically, the formulations are prepared by uniformly and intimately bringing the active compound(s) into association with a liquid carrier, a finely divided solid carrier, or both, and then, if necessary, shaping the product into dosage forms of the desired formulation.

Formulations of the present invention suitable for oral administration may be presented as discrete units such as capsules, cachets, tablets, or lozenges, each containing a predetermined amount of the active ingredient as a powder or granules; or a suspension in an aqueous liquor or a non-aqueous liquid, such as a syrup, an elixir, an emulsion, or a draught.

A tablet may be made by compression or molding, optionally with one or more accessory ingredients. Compressed tablets may be prepared by compressing in a suitable machine, with the active compound being in a free-flowing form such as a powder or granules which optionally is mixed with a binder, disintegrant, lubricant, inert diluent, surface active agent, or discharging agent. Molded tablets comprised of a mixture of the powdered active compound with a suitable carrier may be made by molding in a suitable machine.

A syrup may be made by adding the active compound to a concentrated aqueous solution of a sugar, for example sucrose, to which may also be added any accessory ingredient(s). Such accessory ingredient(s) may include flavorings, suitable preservative, agents to retard crystallization of the sugar, and agents to increase the solubility of any other ingredient, such as a polyhydroxy alcohol, for example glycerol or sorbitol.

Formulations suitable for parenteral administration conveniently comprise a sterile aqueous preparation of the active compound, which preferably is isotonic with the blood of the recipient (e.g., physiological saline solution). Such formulations may include suspending agents and thickening agents and liposomes or other microparticulate systems which are designed to target the compound to blood components or one or more organs. The formulations may be presented in unit-dose or multi-dose form.

Nasal spray formulations comprise purified aqueous solutions of the active compounds with preservative agents and isotonic agents. Such formulations are preferably adjusted to a pH and isotonic state compatible with the nasal mucous membranes.

Formulations for rectal administration may be presented as a suppository with a suitable carrier such as cocoa butter, hydrogenated fats, or hydrogenated fatty carboxylic acids.

Ophthalmic formulations are prepared by a similar method to the nasal spray, except that the pH and isotonic factors are preferably adjusted to match that of the eye.

Topical formulations comprise the active compound dissolved or suspended in one or more media, such as mineral oil, petroleum, polyhydroxy alcohols, or other bases used for topical pharmaceutical formulations.

Transdermal formulations may be prepared by incorporating the active agent in a thixotropic or gelatinous carrier such as a cellulosic medium, e.g., methyl cellulose or hydroxyethyl cellulose, with the resulting formulation then being packed in a transdermal device adapted to be secured in dermal contact with the skin of a wearer.

In addition to the aforementioned ingredients, formulations of this invention may further include one or more accessory ingredient(s) selected from diluents, buffers, flavoring agents, binders, disintegrants, surface active agents, thickeners, lubricants, preservatives (including antioxidants), and the like.

The compounds of formula (I) and pharmaceutically acceptable esters, salts, and other physiologically functional derivatives thereof, may be formed by the exemplary synthetic techniques described in the aforementioned International Publication No. WO93/15062.

The respiratory depression-combating compositions of the present invention may also advantageously attenuate side effects of drug agents other than respiratory depression. For example, fentanyl also induces muscle rigidity through mu receptor activation. Such fentanyl-induced muscle rigidity can be inhibited by a delta agonist compound such as 3-((S)-((2S,5R)-4-Benzyl-2,5-dimethyl-1-piperazinyl)(3-thienyl)methyl)phenol.

Moreover, such combination of mu agonist and delta agonist compounds are synergistic, in selectively antagonizing each other's adverse side effects. The compound 3-((S)-((2S,5R)-4-Benzyl-2,5-dimethyl-1-piperazinyl)(3-thienyl)methyl)phenol at high dosages may induce seizure activity via delta receptor activation. The mu agonist fentanyl, for example, antagonizes such high dose seizure effect, while the delta agonist antagonizes the respiratory depression and muscle rigidity side effects of fentanyl. At the same time, the combination of such agonist compounds results in an additive analgesic effect.

The nausea/vomiting effects of mu-opioid analgesics may also be attenuated by the delta opioid agonist. The combination of mu- and delta-opioid agonists or compounds possessing both mu- and delta-opioid receptor activity may produce less nausea and vomiting as compared to currently used mu-opioid analgesics.

Other side effects of mu-opioid compounds may likewise be ameliorated or even prevented by use of the compounds of the present invention.

In comparison with respiratory depression characteristics of many mu-opioid compounds that are used for mediating analgesia, the compounds of the present invention have substantial advantage. The invention in one aspect contemplates the use of diarylmethylpiperazine compounds for therapeutic intervention which are characterized by a safety ratio, defined as the respiratory depression ED50 concentration of the compound, divided by the analgesia ED50 concentration of the compound, of at least 12 and preferably at least 15. Such diarylmethylpiperazine compounds include the compounds of formula (I) and pharmaceutically acceptable esters and salts thereof, as well as diarylmethylpiperazine compounds generally, e.g., diarylmethylbenzylpiperazine compounds optionally substituted on a phenyl ring of the benzyl group with at least one halogen substituent.

The disease state or physiological condition involved in such therapeutic intervention may be of any suitable type or kind, e.g., a condition such as drug addiction, alcohol addiction, drug overdose, cough, lung edema, gastro-intestinal disorders, arthritis, psoriasis, asthma, inflammatory bowel disease, disorders of respiratory function, functional bowel disease, irritable bowel syndrome, diarrhea, functional distension, pain (e.g., functional pain, trauma pain, etc.), non-ulcerogenic dyspepsia, urogenital tract disorders, organ transplant rejection, skin graft rejection, cardiac disorders, mental disorders, emotional disorders, cognitive disorders, emesis, respiratory depression, acne and skin lesions.

The invention is further illustrated by the following non-limiting examples.

### EXAMPLE 1

The compound of formula (I) was evaluated for in vitro opioid receptor activity in mouse vas deferens (Mouse Vas Deferens ED50). The assay procedure used for such determination of receptor activity is set out below.

In vitro Bioassay:

Vasa deferentia (MVDii, CD-1 strain, Harlan Raleigh N.C.) were removed from mice and suspended between platinum electrodes with 0.5 g of tension in organ bath chambers containing a modified Mg⁺⁺ free Krebs' buffer of the following composition (millimolar): NaCl, 117.5; KCl, 4.75; CaCl₂, 2.6; KH₂PO₄, 1.20; NaHCO₃, 24.5; and glucose, 11. The buffer was saturated with 95% 02/5% CO₂ and kept at 37° C. Tissues were stimulated at supramaximal voltage with 10-Hz pulse trains for 400-msec.; train interval 10 seconds; and 1.0 msec pulse duration at maximal voltage.

The percentage inhibition of the electrically induced muscle contractions was determined for the compounds at varying cumulative concentrations. The ED₅₀ values were extrapolated from curves showing the dose concentration plotted against the response (J. A. H. Lord, A. A. Waterfield, J. Hughes, H. W. Kosterlitz, Nature 267, 495, (1977)). The results are set forth in Table I as shown below:

TABLE I

| Compound | | | |
|---|---|---|---|
| Formula No. | | Mouse Vas Deferens | |
| and | | ED50 (nM) | |
| Designation | Name | μ | δ |
| Formula (II) | 3-((S)-((2S,5R)-4- | 826 | 469 |
| DPI-131 | benzyl-2,5-dimethyl-1- | | |
| | piperazinyl)(3- | | |
| | thienyl)methyl)phenol | | |
| Formula | 3-((R)-((2S,5R)-4- | | 15.7 |
| (VIII) | benzyl-2,5-dimethyl-1- | | |
| DPI-130 | piperazinyl)-3- | | |
| | hydroxybenzyl)-N-(3- | | |
| | fluorophenyl)-N- | | |
| | methylbenzamide | | |

### EXAMPLE 2

Analgesia was assayed in rats using the tail pinch test with simultaneous monitoring of capillary blood gases (PCO₂). During this testing period respiratory depression values were also obtained. Male rats (Wistar Hannover 200-300g) were anesthetized with 2% isoflurane (J. A. Webster, Inc., Sterling, Mass. The femoral artery was cannulated with PE50 tubing for blood sampling. The external jugular vein was also cannulated with Silastic tubing for drug injection. After surgery, anesthetic gases were removed and the rat was allowed to rest in a plastic restrainer for 60 minutes to establish baseline values of blood gases.

The compound DPI-131 was administered intravenously. Nociceptive response and respiratory values were obtained for a 1-2 hour period. The femoral artery cannulation was used to draw arterial blood into a syringe pre-wetted with heparin. Samples were then analyzed with a blood gas analyzer (Ph/Gas Analyzer Synthesis 25 Model, Instrumentation Laboratory) to assess respiratory depression effects. The volume of blood taken each time was 0.15 cc. The syringes were capped immediately and the blood gases analyzed within 5 minutes. The blood exposed to air at the tip of the syringe was expelled. The blood was mixed by gentle inversion and an aliquot of 0.10 cc was injected into the blood gas analyzer.

The gas analyzer was well maintained and operated. Calibrations (low, normal and high) were done at the beginning of every day of testing. The sample lines, co-oximeter and the blood gas electrode were cleaned regularly at the end of every day of testing. Hematocrit calibration (high and low) was scheduled on a weekly basis and tubing, sample and pinch valve were replaced on a monthly basis.

An artery clamp was placed on the tail (one inch from the tip of the tail) for a short duration until an escape response occurred (i.e. tail-flick or vocalization). The escape response latency was recorded by means of a stopwatch. A cutoff time of 20 sec. was used to prevent unnecessary tissue damage. Rats were observed for nociceptive responses of vocalization or painful body movements. The elapsed time to elicit a pain response was recorded as the tail pinch latency in seconds. Blood gases were monitored at approximately the same time points as the tail pinch test. The ED50 values for analgesia potency and respiratory depression were determined to calculate the safety or therapeutic ratio, which is defined as the respiratory depression ED50 divided by analgesia ED50. The analgesic potency (half maximum effective dose, ED50) was determined by the dose at which half of the animals did not show any nociceptive response to the artery clamp pressure with 20 seconds. The results are shown in Table II below.

TABLE 2

| Formula No. | | Respiratory | Analgesia | |
|---|---|---|---|---|
| and | | Depression ED50 | ED50 | |
| Designation | Compound | mg/kg | mg/kg | Safety Ratio |
| Formula (II) | 3-((S)-((2S,5R)-4-Benzyl- | 2.20 | 0.16 | 13.75 |
| DPI-131 | 2,5-dimethyl-1- | | | |
| | piperazinyl)(3- | | | |
| | thienyl)methyl)phenol | | | |
| Formula | 3-((R)-((2S,5R)-4-benzyl- | 2.57 | 0.11 | 23.3 |
| (VIII) | 2,5-dimethyl-1- | | | |
| DPI-130 | piperazinyl)-3- | | | |
| | hydroxybenzyl)-N-(3- | | | |
| | fluorophenyl)-N- | | | |
| | methylbenzamide | | | |

### EXAMPLE 3

3-((S)-((2S,5R)-4-Benzyl-2,5-dimethyl-1-piperazinyl(3-thienyl)methyl)phenol

A solution of 3-bromophenol (500 g, 2.89 mol), tert.-butylchlorodimethylsilane (436 g, 2.89 mol), and imidazole (500 g, 7.22 mol) in 500 mL of dimethylformamide was stirred -overnight at room temperature. The reaction solution was poured into 3000 mL of water and extracted with two 2000 mL portions of diethyl ether. The combined either extracts were dried over sodium sulfate and the solvent removed to give 846 g of 3-(bromophenoxy)-tert.-butyldimethylsilane as a pale yellow liquid. NMR (300 MHz, CDCl₃): δ 0.2 (s, 6H); 1.0 (s, 9H); 6.75 (m, 1H); 7.0 (br s, 1H); 7.1 (m, 2H).

A 12 L, 3-necked round bottom flask was charged with trans-2,5-dimethylpiperazine (767 g, 6.72 mol), which had been recrystallized from toluene to mp=115-119° C., and 600 mL of water. The flask was cooled in an ice bath and a solution of methanesulfonic acid (1290 g, 13.4 mol) in 600 mL of water was added slowly with stirring and cooling to maintain the temperature below 40° C. The solution was cooled to 20° C. and 800 mL of ethanol was added. A 500 mL addition funnel was filled with 60% aqueous potassium acetate from a 2 L reservoir of the solution, and potassium acetate was added to the reaction flask to adjust the pH to 4.0. A second addition funnel was charged with a solution of ethyl chloroformate (642 mL, 6.71 mol) in 360 mL of tetrahydrofuran. The ethyl chloroformate and potassium acetate solutions were simultaneously added dropwise with adjustment of rate to maintain the reaction solution at pH 4.0±0.1, with cooling as necessary to maintain temperature at 25° C. After addition of the ethyl chloroformate was complete, the reaction was stirred for 1 hour with continued addition of potassium acetate solution to maintain a pH of 4.0. The organic solvents were removed by distillation under vacuum. The remaining aqueous solution was washed with 1500 mL of ethyl acetate to remove any bis-carbamate impurity. The ethyl acetate wash was extracted with two 500 mL portions of 1 M hydrochloric acid to recover desired product. The acid extracts were combined with the original aqueous solution and the pH was adjusted to 11 by addition of 10 M sodium hydroxide, with cooling to maintain temperature below 40° C. The aqueous solution was extracted with two 1500 mL portions of ethyl acetate, the combined extracts were dried over magnesium sulfate, and the solvent was removed to give 927 g (74%) ethyl trans-2,5-dimethyl-1-piperazinecarboxylate as a yellow oil.

A mixture of ethyl trans-2,5-dimethyl-1-piperazinecarboxylate (643 g, 3.45 mol), allyl bromide (328 mL, 3.80 mol), and sodium carbonate (440 g, 4.15 mol) in 2500 mL of acetonitrile was heated at reflux for 1.5 hours. The reaction was cooled to room temperature, filtered, and the solvent removed under vacuum. The residue was dissolved in 4000 mL of dichloromethane and washed with two 500 mL portions of 1 M sodium hydroxide. The dichloromethane solution was dried over magnesium sulfate and the solvent was removed to give 630 g (81%) of ethyl trans-4-allyl-2,5-dimethyl-1-piperazinecarboxylate as an oil.

Ethyl trans-4-allyl-2,5-dimethyl-1-piperazinecarboxylate (630 g, 2.78 mol) was added to a solution of 87% potassium hydroxide pellets (2970 g, 46 mol) in 4300 mL of 95% ethanol and heated at reflux for 1.5 hours. Carbon dioxide evolution was observed for the first 0.5-1 hour of heating. The reaction was cooled below reflux temperature and 2000 mL of toluene was carefully added. Ethanol was removed by azeotropic distillation at 105° C., while adding an additional 4000 mL of toluene to the reaction flask during the course of the distillation. After collection of 9000 mL of distillate, the reaction was cooled to 100° C. and 1000 mL of toluene was carefully added. The solution was slowly cooled to 5° C. and maintained at 5° C. for 30 minutes. The solution was filtered, and the filter cake was washed with an additional 1500 mL of toluene. The filtrate was washed with 1000 mL of water, dried over magnesium sulfate, and the solvent was removed to give 296 g (69%) of trans-1-allyl-2,5-dimethylpiperazine as a dark liquid. NMR (300 MHz, DMSO-d6): δ 0.87 (d, J=6.3 Hz, 3H); 0.92 (d, J=6.3 Hz, 3H); 1.63 (t, J=11 Hz, 1H); 2.05 (m, 1H); 2.30 (t, J=11 Hz, 1H); 2.6-2.8 (m, 4H); 3.33 (dd, J₁=5 Hz, J₂=14 Hz, 1H); 5.09 (d, J=8.7 Hz, 1H); 5.13 (d, J=14 Hz, 1H) 5.8 (m, 1H).

Di-p-toluoyl-D-tartaric acid (Schweizerhall, Inc., South Plainfield, N.J.) (1.25 Kg, 3.2 mol) was dissolved in hot (˜60° C.) 95% ethanol (16 L) and racemic trans-1-allyl-2,5-dimethylpiperazine (500 g, 3.2 mol) was added in several portions (caution: exothermic). The hot solution was seeded with crystals of the diastereoisomerically pure salt (obtained from a previous small-scale resolution) and cooled to room temperature over 2-3 hours. The solution was slowly stirred for 2 days at room temperature. The resulting salt was collected by filtration, washed twice with 95% ethanol, and dried under vacuum to give 826.5 g of a white solid (47%). The process was repeated with a second batch of the di-p-toluoyl-D-tartaric acid and racemic trans-1-allyl-2,5-dimethylpiperazine to give 869 g (50%).

The total of 1695 g of salt was divided into three batches and each batch was recrystallized twice in the following fashion. The salt was dissolved in refluxing 95% ethanol (˜2.7 L/100 g of salt), and approximately half of the ethanol was removed by distillation. (Note: vigorous stirring was necessary during distillation to prevent crystallization on the vessel wall.) The hot solution was seeded with crystals of the pure diastereomeric salt, cooled to room temperature, and stirred slowly for 2 days before collecting the salt by filtration. (Note: a subsequent experiment suggested that crystallization time can be reduced from 2 days to 8 hours.) The total amount recovered was 1151 g. The salt was dissolved in 3 L of 2 M aqueous sodium hydroxide, and the aqueous solution was extracted with four 1 L portions of dichloromethane. The organic extracts were combined, dried over sodium sulfate, and solvent removed by rotary evaporation (temperature<20° C.) to give 293 g (29% based on racemic weight) of (2R,5S)-1-allyl-2,5-dimethylpiperazine as a clear oil. [α]_{D}²⁰=−55.1° (abs. ethanol, c=1.2). The trifluoroacetamide of the product was prepared with trifluoroacetic anhydride and analyzed by chiral capillary gas chromatography (Chiraldex B-PH column, 20 m×0.32 mm, Advanced Separation Technologies Inc., Whippany, N.J., 120° C.) indicating an enantiopurity of >99% ee (retention time of desired enantiomer, 11.7 min; other enantiomer, 10.7 min).

3-Phenoxy-tert-butyldimethylsilane magnesium bromide was formed by the slow addition of 2.7 M n-butyllithium in heptane (150 mL, 405 mmol) to a solution of 3-bromophenoxy-tert-butyldimethylsilane (123.44 g, 429 mmol) in 500 mL anhydrous tetrahydrofuran at −70° C. After stirring 45 min. this cold solution was siphoned under nitrogen into a slurry of magnesium bromide etherate (110.62 g, 428 mmol) in 650 mL anhydrous tetrahydrofuran at room temperature, and stirred for 45 min.

Thiophene-3-carboxaldehyde (29.09 g, 259 mmol), benzotriazole (30.91 g, 259 mmol), and (2R,5S)-1-allyl-2,5-trans-dimethylpiperazine (40.01 g, 259 mmol) were dissolved in 250 mL toluene and heated to a gentle reflux. The water-toluene azeotrope was collected in a Dean-Stark trap over the course of 2.5 hours. The remaining solvent was removed under vacuum. The residue was dissolved in 150 mL anhydrous tetrahydrofuran and added to a solution of 3-phenoxy-tert-butyldimethylsilane magnesium bromide in anhydrous tetrahydrofuran (1150 mL, 0.35 M) under a nitrogen atmosphere.

The reaction was stirred at room temperature for 2 hours and then quenched by the addition of 25 mL saturated NH₄Cl solution. Anhydrous magnesium sulfate (˜5 g) and Celite (˜10 g) were added. The mixture was stirred and filtered, and the solvent was removed under reduced pressure. The residue was dissolved in ethyl acetate and washed first with 0.5 N NaOH solution (5×200 mL) and then with brine (1×200 mL). The solution was dried (Na₂SO₄/MgSO₄) and concentrated under reduced pressure.

The dark residue was dissolved in 250 mL anhydrous acetonitrile and tetraethyl-ammonium fluoride dihydrate (72.26 g, 390 mmol) was added. After stirring for 90 min. the reaction was concentrated and the residue was dissolved in 200 mL ethyl acetate. The mixture was extracted with dilute NaHCO₃ solution (3×200 mL) and with water (1×200 mL). The organic layer was diluted with 200 mL diethyl ether and extracted with 10% citric acid solution (8×200 mL). The combined aqueous extracts was adjusted to pH 8.5 using 50% NaOH solution and extracted with dichloromethane (3×200 mL). The solution was dried (Na₂SO₄/MgSO₄) and concentrated under reduced pressure. The resulting tan solid (53.25 g, 155 mmol) was crystallized twice from 225 mL of 2:1/isopropanol: water to yield fluffy, white needle crystals (34.14 g, 99.7 mmol) of 3-((S)-((2S,5R)-4-Allyl-2,5-dimethyl-1-piperazinyl)(3-thienyl)methyl)phenol.

¹H NMR (500 MHz, d₆-DMSO): δ 9.32 (s, 1H), 7.44 (dd, J=3.2, 4.9 Hz, 1H), 7.15 (s, 1H), 7.13 (t, J=8.25 Hz, 1H), 6.98 (d, J=4.9 Hz, 1H), 6.66-6.70 (m, 3H), 5.73-5.81 (m, 1H), 5.15 (d, J=17.1 Hz, 1H), 5.09 (d, J=10.5 Hz, 1H), 5.02 (s, 1H), 3.20 (br d, J=10.2 Hz, 1 H), 2.78 (dd, J=7.3, 7.5 Hz, 1H), 2.68 (dd, J=2.6, 11.3 Hz, 1H), 2.59 (dd, J=1, 9.3 Hz, 1H), 2.44 (br s, 2H), 2.02 (t, J=8.6 Hz, 1H), 1.81 (t, J=8.1 Hz, 1H), 1.09 (d, J=6 Hz, 3H), 0.91 (d, J=6 Hz, 3H).

Calculated for C₂₀H₂₆N₂OS: C, 70.14; H, 7.65; N, 8.18; S, 9.36%. Found: C, 70.19; H, 7.58; N, 8.12; S, 9.33%.

3-((S)-((2S,5R)-4-Allyl-2,5-dimethyl-1-piperazinyl)(3-thienyl)methyl)phenol (8.56 g, 57.1 mmol), N-phenyltrifluoromethanesulfonimide (9.86 g, 27.6 mmol), and triethylamine (8.0 mL, 57.1 mmol) were dissolved in 75 mL dichloromethane and stirred overnight at room temperature under nitrogen. After concentrating under reduced pressure, the residue was dissolved in 150 mL ethyl acetate and washed with Na₂CO₃ solution (3×150 mL), water (1×100 mL), and brine (1×100 mL). The solution was dried (Na₂SO₄/MgSO₄) and concentrated under reduced pressure. The residual oil was purified by chromatography on silica gel (2% NH₄OH in EtOAc/CH₂Cl₂) to give 11.8 g (24.8 mmol) of a viscous, light yellow oil.

The allyl portion was removed using Pd(dba)₂/DPPB in the presence of thiosalicylic acid by the method of Genet [J. P. Genet, S. Lemaire-Audoire, M. Savignac, Tetrahedron Letters, 36, 1267-1270 (1995)]. The reaction was concentrated and the residue was dissolved in 50 mL ethyl acetate and 100 mL diethyl ether. After washing this with Na₂CO₃ solution (3×150 mL) and water (1×100 mL), the organic solution was extracted with 3 N HCl (2×20 mL) and 1 N HCl (2×20 mL). The acidic extract was adjusted to pH 8.5 using NaOH solution and extracted with dichloromethane (3×50 mL). The solution was dried (Na₂SO₄/MgSO₄) and concentrated under reduced pressure. The residual oil was purified by chromatography on silica gel (2% NH₄OH in EtOAc/CH₂Cl₂) to give 8.83 g (20.3 mmol) of a viscous, light amber oil.

The above free amine (1.09 g, 2.5 mmol) was combined with anhydrous sodium carbonate powder (1.50 g, 14.1 mmol), 10 mL anhydrous acetonitrile, and benzyl bromide (0.33 mL, 2.75 mmol). The reaction was stirred overnight at room temperature under nitrogen, and then concentrated under reduced pressure. The residue was suspended in 15 mL ethanol, 10 mL of 10% NaOH solution was added, and the reaction was stirred for 1 hour. The ethanol was removed under vacuum and the residue was partitioned between water and dichloromethane. The solution was adjusted to pH 8.5 using 3 N HCl, separated and extracted again with dichloromethane (2×25 mL). The solution was dried (Na₂SO₄/MgSO₄) and concentrated under reduced pressure. The residual oil was purified by chromatography on silica gel (2% NH₄OH in EtOAc/CH₂Cl₂) to give 0.81 g (1.93 mmol) of 3-((S)-((2S,5R)-4-benzyl-2,5-dimethyl-1-piperazinyl)(3-thienyl)methyl)phenol as a white foam.

¹H NMR (500 MHz, d₆-DMSO): δ 9.33 (s, 1H), 7.45 (dd, J=3, 4.9 Hz, 1H), 7.25-7.36 (m, 4H), 7.17-7.21 (m, 2H), 7.13 (t, J=7.8 Hz, 1H), 6.99 (d, J=4.9 Hz, 1H), 6.66-6.71 (m, 3H), 5.00 (s, 1H), 3.81 (d, J=13.2 Hz, 1H), 3.15 (d, J=12.9 Hz, 1H), 2.65 (dd, J=2.6, 11.2 Hz, 1H), 2.58 (dd, J=2.4, 11 Hz, 1H), 2.42 (br s, 1H), 1.86-1.94 (m, 2H), 1.02 (d, J=5.7 Hz, 3H), 1.01 (d, J=5.7 Hz, 3H).

MS: 393 (M+1, 100%), 189 (32%).

Calculated for C₂₄H₂₈N₂OS.0.3C₄H₈O: C, 72.24; H, 7.31; N, 6.69; S, 7.65%. Found: C, 72.23; H, 7.24; N, 6.74; S, 7.74%.

This material was converted to the hydrochloride salt and precipitated from CH₂Cl₂/Et₂O as an amorphous, white solid.

Calculated for C₂₄H₂₈N₂OS.0.3C₄H₁₀O.1.3HCl: C, 65.49; H, 7.04; N, 6.06; S, 6.94; Cl, 9.97%. Found: C, 65.70; H, 7.34; N, 6.09; S, 6.97; Cl, 9.95%.

### EXAMPLE 4

3-((S)-((2S,5R)-4-(2,6-Difluorobenzyl)-2,5-dimethyl-1-piperazinyl)(3-thienyl)methyl)phenol

The compound of this Example was prepared by following the synthesis procedure as described in Example 3 using 2,6-difluorobenzyl bromide.

The free base was obtained as an off-white foam in 84% yield from 3-((S)-((2S,5R)-4-Allyl-2,5-dimethyl-1-piperazinyl)(3-thienyl)methyl)phenol.

¹H NMR (500MHz, d₆-DMSO): δ 9.31 (s, 1H); 7.45 (dd, J=3.0, 4.9 Hz, 1H); 7.35-7.38 (m, 1H); 7.13 (s, 1H); 7.12 (t, J=7.7 Hz, 1H); 7.05 (t, J=7.8 Hz, 1H); 7.02-7.07 (m, 1H); 6.96 (d, J=4.9 Hz, 1H); 6.66 (br d, J=8.0 Hz, 2H); 6.64 (br s, 1H); 5.02 (s, 1H); 3.83 (d, J=12.6 Hz, 1H); 3.26 (d, J=7.4 Hz, 1H); 2.58-2.62 (m, 2H); 2.50 (m, 1H—obscured by DMSO peak); 2.45 (m, 1H); 2.32 (m, 1H); 1.97 (t, J=9.2 Hz, 1H); 1.77 (m, 1H); 1.05 (d, J=6.0 Hz, 3H); 1.01 (d, J=6.0 Hz, 3H).

This material was converted to the hydrochloride salt and precipitated from CH₂Cl₂/Et₂O as an amorphous, off-white solid.

Calculated for C₂₄H₂₆F₂N₂OS.0.1C₄H₁₀O.0.6H₂O.1.05HCl: C, 60.43; H, 6.07; N, 5.78; S, 6.61; Cl, 7.68%. Found: C, 60.33; H, 6.02; N, 5.71; S, 6.46; Cl, 7.55%.

MS: 429 (M+1, 100%), 189 (11%).

### EXAMPLE 5

3-((R)-((2S,5R)-4-Benzyl-2,5-dimethyl-1-piperazinyl)-3-hydroxybenzyl)-N-(3-fluorophenyl)-N-methylbenzamide

3-Fluoro-N-methylaniline was prepared from 3-fluoroaniline using a modified reductive amination. First, 1-hydroxymethylbenzotriazole was prepared by adding 37% aqueous formaldehyde to benzotriazole at 40° C. in a 1:1 ratio and cooling to room temperature to precipitate the product. After filtration the hydroxymethylbenzotriazole (125 g) was heated to reflux in toluene with 3-fluoroaniline (92.2 g). Water was removed azeotropically using a Dean-Stark trap. After three hours, the mixture was cooled to room temperature, then refrigerated for several hours to complete precipitation. The white crystalline solid was collected by filtration, yielding 174.2 g (86.6%) of 1-((3-fluoroanilino)methyl)-1H-benzotriazole.

1-((3-Fluoroanilino)methyl)-1H-benzotriazole (173.9 g) was slurried in dry tetrahydrofuran. Sodium borohydride (32.5 g) was added portionwise to the mixture at room temperature. After addition was complete, the mixture was heated at reflux for 4 hours. The solution was cooled and poured slowly into 400 mL of 5 M hydrochloric acid with ice and stirred for 1 hour at room temperature. The solution pH was adjusted to 9-10 using 10 M sodium hydroxide solution. The product was extracted using diethyl ether. The ether extracts were washed successively with 1 M sodium hydroxide solution, saturated sodium chloride solution, and water. The organic phase was dried over sodium sulfate and evaporated under reduced pressure to yield 87.5 g (97%) of 3-fluoro-N-methylaniline as a colorless oil. [NMR (200 MHz, DMSO-d₆): δ 2.76 (s, 3H); 3.41 (br s, 1H); 6.59-6.92 (m, 3H); 7.27 (q, J=8.0 Hz, 1H)].

3-Carboxybenzaldehyde was slurried in thionyl chloride (6 mL). A reflux condenser fitted with a calcium chloride drying tube was placed on the flask. The reaction was placed in an oil bath and heated at a bath temperature maintained below 100° C. The mixture was allowed to reflux until a clear solution was obtained and for 5-10 additional minutes before cooling to room temperature. The solution was diluted with anhydrous toluene, and all volatiles were removed under vacuum.

The crude acid chloride was dissolved in dichloromethane and cooled in an ice/water bath. Triethylamine (6 mL) was added dropwise via an addition funnel, followed by N-methyl-3-fluoroaniline (1.83 g) in dichloromethane. The cloudy solution was allowed to warm to room temperature over 1 hour. Water was added and the product was extracted with dichloromethane. The organic layer was washed with water and saturated sodium chloride solution and dried over sodium sulfate, and the solvent was removed under vacuum. N-(3-Fluorophenyl)-3-formyl-N-methylbenzamide (3.20 g) was obtained as a light golden oil (93% unchromatographed yield).

[NMR (300 MHz, DMSO-d6): δ 3.38 (s, 3H); 6.94-7.02 (m, 2H); 7.18-7.29 (m, 2H); 7.46 (t, J=7.7 Hz, 1H) 7.55 (d, J=7.6 Hz, 1H); 7.81 (m, 2H); 9.90 (s, 1H)].

2R,5S-1-allyl-2,5-dimethylpiperazine (as prepared in Example 3, 1.28 g, 8.3 mMol.), benzotriazole (1.00 g, 8.4 mMol., 1.01 eq., Aldrich), and N-(3-fluorophenyl)-3-formyl-N-methylbenzamide (2.14g, 8.3 mMol.) were mixed in 80 mL of dry toluene with one drop of triethylamine. The mixture was placed in an oil bath maintained below 140° C. (bath temperature.

The flask was attached to a Dean-Stark trap and reflux condenser to allow the azeotropic removal of water. The mixture was refluxed for 2-3 hours, under a nitrogen atmosphere, then the majority of the toluene was removed under reduced pressure. The crude adduct was used in the following procedure without isolation.

The crude benzotriazole adduct was dissolved in ˜10 mL of tetrahydrofuran and added to a solution of 3-phenoxy-tert-butyldimethylsilane magnesium bromide (as prepared in Example 3, 1.75 equiv.) via a double-ended needle. After stirring under nitrogen at room temperature for 2 hours, the reaction was quenched with 3-4 mL of saturated ammonium chloride solution. Having stirred this for about half an hour, a generous amount of anhydrous magnesium sulfate was added. Filtering and concentrating the solution under reduced pressure gave the crude silyl ether contaminated with benzotriazole by-product. This residue was dissolved in ethyl acetate and extracted with 10% aqueous NaOH solution three times to remove most of the benzotriazole. The organic layer was washed with saturated sodium chloride solution, dried over sodium sulfate/magnesium sulfate, and the ethyl acetate was removed under reduced pressure.

The t-butyldimethylsilyl protecting group was removed by dissolving the residue in 40 mL of tetrahydrofuran and adding 40 mL of 3N aqueous HCl at room temperature. The solution warmed upon acid addition. The mixture was stirred for 90 minutes at room temperature. The reaction was concentrated under reduced pressure to remove most of the organic solvent. The residue was partitioned between water and a solution of diethyl ether:ethyl acetate/3:2. The acidic aqueous layer was extracted twice with a solution of diethyl ether:ethyl acetate/3:2.

The aqueous layer was adjusted to pH=2 using aqueous NaOH solution, at which point cloudiness persisted and a dark oil began to precipitate. Methylene chloride (˜100 mL) was added and stirred briskly. This was separated and the aqueous layer was again washed with more methylene chloride. The combined organic extract was partitioned with water, and while stirring vigorously was adjusted to pH=9 using aqueous NaOH solution. This was then separated and the aqueous layer was again washed with more methylene chloride.

The combined extract was dried over sodium sulfate/magnesium sulfate, and the solvent was evaporated under reduced pressure. The crude material was chromatographed on silica gel column (roughly 20-25 g of silica gel per gram of crude material) eluting first with methylene chloride, then with 20% ethyl acetate in methylene chloride to remove the less polar contaminant. Then, the column was eluted with a solution of ethyl acetate containing 2% ammonium hydroxide (solution A) in a gradient with methylene chloride (solution B), quickly increasing in polarity from 25% to 100% (solution A in B).

The desired fractions were combined and the solvent was removed under reduced pressure. A 10:1 mixture of diastereomers (approx. 2.6 g) was obtained. Pure product was obtained by crystallization from a hot solution of ethyl acetate (5-10 mL) followed by slow addition of heptane (10-20 mL) and gradual cooling to give 1.78 g of (+)-3-((αR)-α-((2S,5R)-4-allyl-2,5-dimethyl-1-piperazinyl)-3-hydroxybenzyl)-N-(3-fluorophenyl)-N-methylbenzamide as an off-white crystalline solid (m.p.=144-145° C.) with >98% isomeric purity (as determined by NMR). NMR (200 MHz, DMSO-d₆): δ 0.84 (d, J=6.0 Hz, 3H); 0.97 (d, J=5.9 Hz, 3H); 1.69 (dd, J₁=7.7 Hz, J₂=10.7 Hz, 1H); 2.01 (dd, J₁=7.4 Hz, J₂=10.7 Hz, 1H); 2.28 (br. d, J=8.3 Hz, 1H); 2.40-2.52 (m, 2H); 2.67 (br d, J=10.5 Hz, 1H); 2.82 (dd, J₁=7.6 Hz, J₂=13.2 Hz, 1H); 3.17 (br. d, J=14.0 Hz, 1H); 3.34 (s, 3H); 4.80 (s, 1H); 5.10 (d, J=10.1 Hz, 1H); 5.17 (d, J=17.3 Hz, 1H); 5.70-5.84 (m, 1H); 6.42 (d, J=7.1 Hz, 1H); 6.56 (s, 1H); 6.65 (d, J=8.3 Hz, 1H); 6.90-7.32 (m, 9H); 9.31 (s, 1H). Mass spectrum (C₁-CH₄) m/z: 488 (m+1, 100%), 334 (39%), 153 (87%).

[α]_{D}²⁰=+4.9° (abs. ethanol, c=1.2).

Next, 3-((R)-((2S,5R)-4-Allyl-2,5-dimethyl-1-piperazinyl)-3-hydroxybenzyl)-N-(3-fluorophenyl)-N-methylbenzamide (4.88 g, 10 mmol), N-phenyltrifluoromethane-sulfonimide (3.82 g, 10.7 mmol), and triethylamine (3.1 mL, 22 mmol) were dissolved in 75 mL dichloromethane and stirred overnight at room temperature under nitrogen. After concentrating under reduced pressure, the residue was dissolved in 100 mL ethyl acetate and washed with Na₂CO₃ solution (3×100 mL), water (1×100 mL), and brine (1×100 mL). The solution was dried (Na₂SO₄/MgSO₄) and concentrated under reduced pressure. The residual oil was purified by chromatography on silica gel (2% NH₄OH in EtOAc/CH₂Cl₂) to give 6.1 g (9.8 mmol) of a viscous, golden yellow oil.

The allyl portion was removed using Pd(dba)2/DPPB in the presence of thiosalicylic acid by the method of Genet [J. P. Genet, S. Lemaire-Audoire, M. Savignac, Tetrahedron Letters, 36, 1267-1270 (1995)]. The reaction was concentrated and the residue was dissolved in 50 mL ethyl acetate and 100 mL diethyl ether. After washing this with NaCO₃ solution (3×100 mL) and water (1×100 mL), the organic solution was extracted with 3 N HCl (3×20 mL) and 1 N HCl (1×20 mL). The acidic extract was adjusted to pH 8.5 using NaOH solution and extracted with dichloromethane (3×25 mL). The solution was dried (Na2SO₄/MgSO₄) and concentrated under reduced pressure. The residual oil was purified by chromatography on silica gel (2% NH₄OH in EtOAc/CH₂Cl₂) to give 4.44 g (7.6 mmol) of a viscous, deep amber-orange colored oil.

The above free amine (0.867 g, 1.5 mmol) was combined with anhydrous sodium carbonate powder (0.81 g, 7.64 mmol), 10 mL anhydrous acetonitrile, and benzyl bromide (0.20 mL, 1.68 mmol). The reaction was stirred overnight at room temperature under nitrogen, and then concentrated under reduced pressure. The residue was suspended in 15 mL ethanol, 10 mL of 10% NaOH solution was added, and the reaction was stirred for 30 minutes. The ethanol was removed under vacuum and the residue was partitioned between water and dichloromethane. The solution was adjusted to pH 8.5 using 3 N HCl, separated and extracted again with dichloromethane (2×25 mL). The solution was dried (NaSO₄/MgSO₄) and concentrated under reduced pressure. The residual oil was purified by chromatography on silica gel (2% NH₄OH in EtOAc/CH₂Cl₂) to give 0.44 g (0.82 mmol) of the desired product as a white foam. ¹H NMR (500 MHz, d₆-DMSO): δ 9.32 (s, 1H), 7.19-7.30 (m, 10H), 7.05-7.09 (m, 2 H), 6.98 (dt, J=2.3, 8.4 Hz, 1H), 6.89 (dd, J=1.0, 8.0 Hz, 1H), 6.63 (dd, J=1.0, 8.0 Hz, 1H), 6.57 (br s, 1H), 6.43 (d, J=7.4 Hz, 1H), 4.77 (br s, 1H), 3.77 (d, J=13.7 Hz, 1H), 3.36 (s, 3H), 3.21 (d, J=13.7 Hz, 1H), 2.59 (d, J=9.0 Hz, 1H), 2.50 (m, 2H obscured by DMSO peak), 2.35 (d, J=9.0 Hz, 1H), 1.92 (dd, J=7.4, 10.9 Hz, 1H), 1.74 (dd, J=7.4, 10.9 Hz, 1H), 0.99 (d, J=6.1 Hz, 3H),0.92(d, J=6.1 Hz, 3H).

MS: 538 (M+1, 100%), 334 (20%).

Calculated for C₃₄H₃₆FN₃O₂.0.15C₄H₈O₂.0.06CH₂Cl₂: C, 74.88; H, 6.77; N, 7.56; F, 3.42%. Found: C, 74.72; H, 6.96; N, 7.38; F, 3.79%.

This material was converted to the hydrochloride salt and precipitated from CH₂Cl₂/Et₂O as an amorphous, off-white solid.

Calculated for C₃₄H₃₆FN₃O₂.0.1C₄H₁₀O.1.1HCl0.1H₂O: C, 70.39; H, 6.58; N, 7.16; Cl, 6.64%. Found: C, 70.41; H, 6.56; N, 7.13; Cl, 6.60%.

### EXAMPLE 6

3-((R)-((2S,5R)-2,5-Dimethyl-4-(4-fluorobenzyl)-1-piperazinyl)-3-hydroxybenzyl)-N-(3-fluorophenyl)-N-methylbenzamide

The compound of this Example was prepared by following the synthesis procedure as described in Example 5 using 4-fluorobenzyl bromide.

The free base was obtained as an off-white foam in 58% yield from 3-((R)-((2S,5R)-4-Allyl-2,5-dimethyl-1-piperazinyl)-3-hydroxybenzyl)-N-(3-fluorophenyl)-N-methylbenzamide.

¹H NMR (600 MHz, d₆-DMSO): δ 9.29 (s, 1H), 7.16-7.29 (m, 7H), 7.02-7.10 (m, 4 H), 6.97 (dt, J=2.3, 8.4 Hz, 1H), 6.88 (dd, J=1.2, 8.0 Hz, 1H), 6.61 (dd, J=1.2, 8.0 Hz, 1H), 6.55 (br s, 1H), 6.42 (br d, J=7.3 Hz, 1H), 4.74 (br s, 1H), 3.71 (br d, J=13.0 Hz, 1H), 3.34 (s, 3H), 3.19 (br d, J=13.0 Hz, 1H), 2.56 (d, J=9.0 Hz, 1H), 2.48 (m, 2H—obscured by DMSO peak), 2.32 (d, J=9.0 Hz, 1H), 1.90 (dd, J=7.2, 11.1 Hz, 1H), 1.72 (dd, J=7.2, 11.1 Hz, 1H), 0.97 (d, J=6.1 Hz, 3H), 0.90 (d, J=6.1 Hz, 3H).

MS: 556 (M+1, 100%), 334 (26%).

This material was converted to the hydrochloride salt and precipitated from CH₂Cl₂/Et₂O as an amorphous, off-white solid.

Calculated for C₃₄H₃₅F₂N₃O₂.0.5H₂O.0.95HCl: C, 68.14; H, 6.21; N, 7.01; Cl, 5.62%. Found: C, 68.17; H, 6.27; N, 6.91; Cl, 5.63%.

### EXAMPLE 7

4-((alpha-R)-alpha-((2S,5R)-2,5-Dimethyl-4-(2-fluorobenzyl)-1-piperazinyl)-3-hydroxybenzyl)-N,N-diethylbenzamide

4-((alpha-R)-alpha-((2S,5R)-4-Allyl-2,5-Dimethyl-1-piperazinyl)-3-hydroxybenzyl)-N,N-diethylbenzamide hydrochloride (M. J. Bishop and R. W. McNutt. Bioorganic and Med. Chem. Lett. 5, 1311-1314, 1995) (0.97 g, 2.0 mmol) was dissolved in methylene chloride (10 mL) and triethylamine (0.919 mL, 0.667 g, 6.6 mmol) added. N-Phenyl bis(trifluoromethanesulfonimide (0.785 g, 2.2 mmol) was added and the reaction mixture sealed under nitrogen and stirred at room temperature overnight. The reaction mixture was evaporated to dryness, the residue dissolved in ethyl acetate (20 mL), and the solution extracted with 5% sodium carbonate solution (2×15 mL). The organic layer was separated, dried over anhydrous sodium sulfate and evaporated to yield a viscous amber oil. The residue was dissolved in methylene chloride (5 mL) and applied to a column of silica gel (4×30 cm), and eluted with ethanol/methylene chloride (2:98 v/v). Pure fractions containing desired product, as evidenced by t.l.c. (silica gel, EM60F₂₅₄, 2% NH₄OH in ethyl acetate, Rf =0.78) were evaporated to dryness to yield 4-((alpha-R)-alpha-((2S,5R)-4-Allyl-2,5-dimethyl-1-piperazinyl)-3-trifluoromethylsulfonyloxybenzyl) N,N-diethylbenzamide (0.72 g) as a yellow/amber oil.

¹H NMR (CDCl3, 500 MHz); 6 1.00 (d, J=6.2 Hz, 3H); 1.12 (br m, 3H); 1.21 (d, J=6.1 5 Hz, 3H); 1.25 (br m, 3H); 1.83 (t, J=10.6 Hz, 1H); 2.60 (m, 3H); 2.91 (dd J=11.4, 2.7, 1H); 3.02 (m, 1H); 3.18 (br s, 2H); 3.28 (br m, 2H); 3.46 (dd,.J=13.7, 5.5 Hz, 1H); 3.55 (br m, 2H); 5.25 (m, 2H); 5.31 (s, 1H); 5.88 (m, 1H); 7.02 (d, J=7.7 Hz, 1H); 7.05 (s, 1H); 7.23 (m, 2H); 7.32 (d, J=8.1 Hz, 2H); 7.40 (d, J=8.1 Hz, 2H); 7.46 (t, J=7.9 Hz, 1H).

A solution of 4-((α-R)-α-((2S,5R)-4-Allyl-2,5-dimethyl-1-piperazinyl)-3-trifluoromethylsulfonyloxybenzyl)-N,N-diethylbenzamide (0.72 g, 1.286 mmol) and thiosalicylic acid (234.7 mg, 1.522 mmol) in anhydrous tetrahydrofuran (4 mL) was stirred under nitrogen for 3 h at room temperature with a catalyst solution prepared by dissolution of bis(dibenzylidineacetone)palladium (36.46 mg, 0.0634 mmol) and 1,4-bis(diphenylphosphino)butane (27.04 mg, 0.0634 mmol) on tetrahydrofuran (0.5 mL). The reaction mixture was evaporated to dryness, the residue dissolved in a mixture of ethyl acetate/ether (1:3, 20 mL) and extracted with 5% sodium carbonate solution (2×15 mL). The organic layer was diluted with two volumes of pentane and extracted with 3M-hydrochloric acid (5×4 mL). The aqueous solution was adjusted to pH 9-10 with concentrated ammonia solution and extracted with methylene chloride (3×10 mL).

The combined organic extracts were dried over anhydrous sodium sulfate and evaporated to yield 4-((α-R)-α-((2S,5R)-2,5-dimethyl-1-piperazinyl)-3-trifluoromethyl-sulfonyloxybenzyl)-N,N-diethylbenzamide as a brittle pale yellow foam (0.63 g). The product showed a single spot on thin layer chromatography (silica gel, EM60F₂₆₄, 2% NH4OH in ethyl acetate, Rf =0.33). ¹H NMR (CDCl₃, 500 MHz); 6 0.95 (d, J=6 Hz, 3H); 1.13 (br m, 3H); 1.20 (d, J=6.1 Hz, 3H);

1.26 (br m, 3H); 1.50 (t, J=9.7 Hz, 1H); 2.31 (m, 1H); 2.64 (dd J=11.3, 2.5, 1H); 2.71 (m, 1H); 2.95 (m, 1H); 3.29 (br m, 2H); 3.56 (br m, 2H); 5.43 (s, 1H); 7.04 (m, 1H); 7.21 (d, J=7.7, 1H);

7.24 (dd, J=8.2, 2.2 Hz, 1H); 7.34 (d, J=8.2 Hz, 2H); 7.42 (d, J=8.1 Hz, 2H); 7.48 (t, J=8 Hz, 1H);

4-((α-R)-α-((2S,5R)-2,5-Dimethyl-1-piperazinyl)-3-trifluoromethylsulfonyloxy-benzyl)-N,N-diethylbenzamide (527.6 mg, 1.0 mmol) was dissolved in acetonitrile (4.0 mL) and sodium iodide (15 mg, 0.1 mmol) added. The suspension was stirred during the addition of triethylamine (500 μL (363 mg), 3.59 mmol), followed by 2-fluorobenzyl bromide (241 μL (378 mg), 2.0 mmol). The reaction mixture was sealed under nitrogen and stirred overnight at room temperature.

The reaction mixture was evaporated to dryness and partitioned between ethyl acetate (10 mL) and saturated aqueous sodium bicarbonate solution (2.5 mL). The supernatant organic layer was removed, and the aqueous portion washed with ethyl acetate (3×10 mL). The combined organic extract and washings were dried over anhydrous sodium sulfate and evaporated to a golden oil. The residue was dissolved in ethyl acetate (7 mL), applied to a pre-packed (Biotage) column and eluted with ethyl acetate.

Pure fractions containing desired product, as evidenced by t.l.c. (silica gel, EM60F₂₆₄, 100% ethyl acetate, R_{f}=0.77), were evaporated to dryness to yield the intermediate 4-((α-R)-α-((2S,5R)-2,5-Dimethyl-4-(2-fluorobenzyl)-1-piperazinyl)-3-trifluoromethylsulfonyloxybenzyl)-N,N-diethylbenzamide (610 mg), as a yellow oil which was used without further purification. The oil was dissolved in ethanol (7 mL) and aqueous 2.5 M (10%) sodium hydroxide solution (5 mL, 12.5 mmol) was added. The reaction mixture was set aside at room temperature for 5 h, then the ethanol removed by evaporation. The oily suspension of the sodium salt was clarified by the addition of water (5 mL), and the pH of the solution adjusted to 9-10 by the passage of gaseous carbon dioxide (from dry ice). The copious white precipitate was washed well with water and dried under vacuum (2 mm Hg) at room temperature overnight to yield 4-((α-R)-α-((2S,5R)-2,5-dimethyl-4-(2-fluorobenzyl)-1-piperazinyl)-3-hydroxybenzyl)-N,N-diethylbenzamide as a white solid (431 mg, 85.6%).

Calc. for C₃₁H₃₈FN₃O₂: C, 73.93; H, 7.61; N, 8.34; F, 3.77. Found C, 73.96; H, 7.67; N, 8.29; F, 3.75%. ¹H NMR (CDCl3, 300 MHz); 6 1.05 (d, J=6.1 Hz, 3H); 1.09 (d, J=6 Hz, 3H); 1.12 (br m, 3H); 1.24 (br m, 3H); 1.96 (t, J=10 Hz, 1H); 2.07 (t, J=10 Hz, 1H); 2.56 (br m, 2H); 2.60 (d, J=11 Hz, 1H); 2.72 (d, J=11 Hz, 1H); 3.29 (br m, 2H); 3.36 (d, J=14 Hz, 1H); 3.55 (br m, 2H); 3.89 (d, J=14 Hz, 1H); 5.13 (s, 1H); 6.57 (s, 1H); 6.66 (d, J=10 Hz, 2H); 7.00 (t, J=9 Hz, 1H); 7.07 (t, J=7.5 Hz, 1H); 7.10 (t, J=8 Hz, 1H); 7.20 (m, 1H); 7.27 (d, J=8 Hz, 2H); 7.38 (t, J=7 Hz, 1H); 7,43 (d, J=7 Hz, 2H).

### EXAMPLE 8

4-((α-R)-α-((2S,5R)-2,5-Dimethyl-4-(4-bromobenzyl)-1-piperazinyl)-3-hydroxybenzyl)-N,N-diethylbenzamide

4-((α-R)-α-((2S,5R)-2,5-Dimethyl-4-(4-bromobenzyl)-1-piperazinyl)-3-hydroxybenzyl)-N,N-diethylbenzamide was prepared in similar fashion to Example 7 from 4-((α-R)-α-((2S,5R)-2,5-dimethyl-1-piperazinyl)-3-trifluoromethylsulfonyloxybenzyl)-N,N-diethylbenzamide and 4-bromobenzyl bromide in 88.4% yield.

Calc. for C₃₁H₃₈BrN₃O₂: C, 65.95; H, 6.78; N, 7.44; Br, 14.15. Found C, 65.97; H, 6.83; N, 7.39; Br, 14.05%

¹H NMR (CDCl3, 300 MHz); δ 1.05 (m, 6H); 1.12 (br m, 3H); 1.24 (br m, 3H); 1.97 (m, 2H); 2.54 (br m, 1H); 2.59 (br m, 1H) 2.61 (d, J=11.5 Hz, 1H); 2.64 (d, J=11 Hz, 1H); 3.14 (d, J=13.2 Hz, 1H); 3.28 (br m, 2H); 3.54 (br m, 2H); 3.84 (d, J=12.8 Hz, 1H); 5.01 (s, 1H); 6.62 (s, 1H); 6.70 (m, 2H); 7.13 (t, J=7.8 Hz, 1H); 7.17 (d, J=8.1 Hz); 7.27 (d, J=8.1 Hz, 2H); 7,40 (d, J=8.3 Hz, 2H); 7.43 (d, J=8 Hz, 2H).

### EXAMPLE 9

4-((α-R)-α-((2S,5R)-2,5-Dimethyl-4-(2-chlorobenzyl)-1-piperazinyl)-3-hydroxybenzyl)-N,N-diethylbenzamide was prepared in similar fashion to Example 7 from 4-((α-R)-α-((2S,5R)-2,5-dimethyl-1-piperazinyl)-3-trifluoromethylsulfonyloxybenzyl)-N,N-diethylbenzamide and 2-chlorobenzyl bromide in 86% yield. Calc. for C₃₁H₃₈ClN₃O₂0.5H₂O: C, 70.37; H, 7.43; N, 7.94. 30 Found C, 70.66; H, 7.25; N, 7.66%.

¹H NMR (CDCl₃, 300 MHz); 8 1.05 (br m, 6H); 1.12 (br m, 3H); 1.24 (br m, 3H); 1.97 (m, 1H); 2.09 (m, 1H); 2.61 (br m, 2H) 2.74 (d, J=9.3 Hz, 1H); 3.30 (br m 2H); 3.15 (d, J=14.7 Hz, 1H); 3.54 (br m, 2H); 3.90 (d, J=14.1 Hz, 1H); 4.96 (s, 1H); 6.63 (s, 1H); 6.69 (d, J=7 Hz, 2H); 7.15 (m, 3H); 7.29 (m, 3H); 7.45 (m, 3H).

### EXAMPLE 10

3-((S)-((2S,5R)-2,5-Dimethyl-4-(4-(iodobenzyl)-1-piperazinyl)3-pyridylmethyl)phenol

Toluene (400 mL) was charged into a round-bottom flask fitted with a reflux condenser, Dean-Stark trap, nitrogen inlet and thermometer. The apparatus was sealed under nitrogen, the toluene heated to boiling and 20 mL of the solvent removed by distillation. A solution of (2R,5S)-1-allyl-2,5-dimethylpiperazine (7.713 g, 50 mmol) in toluene (20 mL) was added and a further 20 mL of toluene removed by distillation. Benzotriazole (5.96 g, 50 mmol) was added as a 1:4 slurry in toluene, and a further 20 mL of toluene removed by distillation. Pyridine-3-aldehyde (5.36 g, 50 mmol) was added, followed by sulfuric acid (53 μL, ˜1.0 mmol).

The reaction mixture was heated under nitrogen for 2 h, during which time the Dean-Stark trap was emptied several times, collecting a total of μ80 mL of turbid distillate and liquid water. As the distillate cleared, a gentle flow of nitrogen gas was applied to the apparatus to aid in the removal of water, and periodically the condenser was removed and washed successively with ethanol and toluene to remove accumulated water droplets. The reaction mixture, which had been maintained below 115° C. during the reaction period (3 h) was cooled to room temperature and evaporated to ˜100 mL. The residue was dissolved in anhydrous tetrahydrofuran (300 mL) and a solution of a Grignard reagent, prepared by sonication of 3-bromophenoxy-t-butyldimethylsilane (28.7 g, 100 mmol) in anhydrous tetrahydrofuran (200 mL) with magnesium turnings (2.67 g, 110 mmol) under nitrogen for 45 min, was added over via a double ended needle over 10 min. The gelatinous suspension was stirred for a further 90 min, then the reaction was quenched by the addition of saturated aqueous ammonium chloride solution (22 mL).

The suspension was stirred for a further 30 min, anhydrous magnesium sulfate (30 g) added, and stirring continued for a further 15 min. The suspension was filtered, the filter cake washed with tetrahydrofuran (100 mL), and the combined filtrate and washings dried over anhydrous sodium sulfate. The solution was evaporated, the residue dissolved in ethyl acetate, and the solution washed successively with aqueous 1.0-M sodium hydroxide (3×200 mL), water (2×200 mL) and saturated aqueous sodium chloride solution. The organic layer was separated, dried over anhydrous sodium sulfate and evaporated to an oil.

The residue was dissolved in methylene chloride (20 mL), applied to a column of silica gel (8×50 cm), and eluted with methylene chloride:ethanol:NH₄OH (95:5:1, 4.0 L) followed by methylene chloride:ethanol:NH₄OH (92.5:7.5:1, 2.0 L). Pure fractions containing desired product, as evidenced by t.l.c. (silica gel, EM60F₂₅₄, methylene chloride:ethanol:NH₄OH (92.5:7.5:1, R_{f}=0.49) were evaporated to dryness to yield 3-((S)-((2S,5R)-4-allyl-2,5-dimethyl-1-piperazinyl)3-pyridylmethyl)phenol as a brittle beige foam.

(7.93 g, 47.4%) ¹H NMR (CDCl₃, 300 MHz); δ 0.97 (d, J=6.3 Hz, 3H); 1.11 (d, J=6.1 1 Hz, 3H);1.88 (m, 1H); 2.12 (m, 1H); 2.43 (m, 2H); 2.58 (m, 1H); 2.80 (dd, J 11.6, 2.9, 1H); 2.86 (m, 1H); 3.39 (dd, J=13.8, 5.2 Hz, 1H); 5.18 (m, 3H); 5.82 (m, 1H); 6.47 (s, 1H); 6.59 (d, J=7.6 Hz, 1H); 6.71(dd, J=8, 1.7 Hz, 1H); 7.14 (t, J=7.8 Hz, 1H); 7.26 (dd, J=7.6, 4.8 Hz, 1H); 7.90 (d, J=7.9 Hz, 1H); 8.43(d, J=4 Hz, 1H); 8.53 (d, J=0.9 Hz, 1H).

3-((S)-((2S,5R)-4-allyl-2,5-dimethyl-1-piperazinyl)3-pyridylmethyl)phenol (3.45 g, 10 mmol) was dissolved in methylene chloride and triethylamine (5.24 mL, 3.806 g, 37.6 mmol) was added. To the stirred solution was added N-phenyl-bis(trifluoromethanesulfonimide) (5.36 g, 15 mmol) and the solution stirred under nitrogen overnight. The reaction mixture was evaporated to an oil, which was dissolved in ethyl acetate and extracted with 5% aqueous sodium carbonate solution (3×15 mL), then washed with saturated sodium chloride solution. The organic layer was dried over sodium sulfate and the solvent removed in vacuo to yield a viscous amber oil. The residue was dissolved in ethyl acetate and applied to a silica gel column (5×40 cm) and eluted with ethyl acetate. Pure fractions containing desired product, as evidenced by t.l.c. (silica gel, EM60F₂₆₄, 100% ethyl acetate, R_{f}=0.51) were evaporated to dryness to yield O-trifluoromethylsulfonyl-3-((S)-((2S,5R)-4-allyl-2,5-dimethyl-1-piperazinyl)3-pyridylmethyl)phenol as a yellow oil (83.9%).

¹H NMR (CDCl₃, 300 MHz); δ 0.97 (d, J=6.1 Hz, 3H); 1.18 (d, J=6.1 Hz, 3H);1.78 (m, 1H); 2.15 (m, 1H); 2.53 (m, 2H); 2.83 (m, 2H) 3.34 (dd, J=13.7, 5.5 Hz, 1H); 5.14 (m, 2H); 5.19 (m, 1H); 5.33 (s, 1H); 5.82 (m, 1H); 7.05 (s, 1H); 7.22 (m, 3H); 7.43 (m, 1H); 7.66 (d, J=7.9 Hz, 1H); 8.48 (m, 1H); 8.65(m, 1H).

A solution of O-trifluoromethylsulfonyl-3-((S)-((2S,5R)-4-allyl-2,5-dimethyl-1-piperazinyl)3-pyridylmethyl)phenol (4.00 g, 8.52 mmol) and thiosalicylic acid (1.715 g, 11.13 mmol) in anhydrous tetrahydrofuran (25 mL) was stirred under nitrogen for 2 h at room temperature with a catalyst solution prepared by dissolution of bis(dibenzylidineacetone)palladium (266 mg, 0.463 mmol) and 1,4-bis(diphenylphosphino)butane (198 mg, 0.464 mmol) in tetrahydrofuran (4 mL). The reaction mixture was evaporated to dryness, the residue dissolved in a mixture of ethyl acetate/ether (1:3, 50 mL) and extracted with 5% sodium carbonate solution (3×20 mL). The organic layer was diluted with an equal volume of pentane and extracted with 3M-hydrochloric acid (5×4 mL). The aqueous solution was adjusted to pH 9-10 with saturated sodium carbonate solution and extracted with methylene chloride (3×20 mL).

The combined organic extracts were dried over anhydrous sodium sulfate and evaporated to yield O-trifluoromethylsulfonyl-3-((S)-((2S,5R)-2,5-dimethyl-1-piperazinyl)3-pyridylmethyl)-phenol as a yellow oil (3.56 g, 97.3%). The product showed a single spot on thin layer chromatography (silica gel, EM60F₂₅₄, 4% NH4OH/10% ethanol in ethyl acetate, Rf =0.35). ¹H NMR (CDCl₃, 300 MHz); δ 1.00 (d, J=6.4 Hz, 3H); 1.21 (d, J=6.1 Hz, 3H);1.63 (t, J=11 Hz, 1H); 2.44 (m, 1H); 2.66 (m, 3H); 3.02 (m, 2H); 5.44 (s, 1H); 5.82 (m, 1H); 7.03 (s, 1H); 7.25 (m, 3H); 7.48 (m, 1H); 7.66 (d, J=8.1 Hz, 1H); 8.50 (d, J=4.4 Hz, 1H); 8.64 (s, 1H).

Using alkylation and deprotection procedures similar to those of Example 7, 3-((S)-((2S,5R)-2,5-Dimethyl-4-(4-(iodobenzyl)-1-piperazinyl)3-pyridylmethyl)phenol was prepared in 38% yield from O-trifluoromethylsulfonyl-3-((S)-((2S,5R)-2,5-dimethyl-1-piperazinyl)-3-pyridylmethyl)phenol and 4-iodobenzyl bromide.

Calc. for C₂₅H₂₈IN₃O0.2H₂O: C, 58.08; H, 5.44; N, 8.13; I, 24.55. Found C, 58.12; H, 5.46; N, 8.00; I, 24.44%

¹H NMR (CDCl₃, 600 MHz); δ 1.00 (d, J=6.2 Hz, 3H); 1.08 (d, J=6.1 Hz, 3H); 1.94 (m, 2H); 2.45 (d, J=11.4 Hz, 1H); 2.49 (br m, 1H); 2.58, br m, 1H) 2.64 (dd, J=11.1, 2.3 Hz, 1H); 3.11 (d, J=13.3 Hz, 1H); 3.84 (d, J=13.3 Hz, 1H); 5.11 (s, 1H); 6.54 (s, 1H); 6.73 (d, J=7.5 Hz, 1B); 6.77 (dd, J=8, 2 Hz, 1H); 7.04 (d, J=8.2 Hz, 2H); 7.19 (t, 7.8 Hz, 1H); 7.25 (m, 1H); 7.60 (d, 8.2 Hz, 2H); 7.89(d, J=8 Hz, 1H); 8.42 (d, J=4.2 Hz, 1H); 8.52 (d, J=1.1 Hz, 1H).

### EXAMPLE 11

3-((S)-((2S,5R)-4-(4-fluorobenzyl)-2,5-dimethyl-1-piperazinyl)(3-thienyl)methyl)phenol

A suspension of 3-((S)-((2S,5R)-4-allyl-2,5-dimethyl-1-piperazinyl)(3-thienyl)methyl)phenol (prepared as in Example 3, 3.192 g, 9.32 mmol), chlorotris(triphenylphosphine)rhodium (I) (1.0 g, 1.08 mmol) in 4:1 acetonitrile/water (100 ml) was gently refluxed for 1 hr. Acetonitrile was slowly distilled off while 100 ml of 4:1 acetonitrile/water was added into the reaction mixture in such a rate to maintain the continuous distillation. Volume was reduced to ˜40 mL over 3 hrs. The resulting dark brown suspension was evaporated to a fluffy solid which was purified by column chromatography eluting with 5% and then 10% ammoniated MeOH in CH₂Cl₂ to give 3-((S)-((2S,5R)-2,5-dimethyl-1-piperazinyl)(3-thienyl)methyl)phenol as a dark brown solid, 2.34 g (83% yield).

To a solution of 3-((S)-((2S,5R)-2,5-dimethyl-1-piperazinyl)(3-thienyl)methyl)phenol (362.93 mg, 1.2 mmol) and 4-fluorobenzaldehyde (223 mg, 1.8 mmol) in anhydrous tetrahydrofuran (8 ml) was added 100 mg of acetic acid at RT and stirred under nitrogen at RT for 30 min. Sodium triacetoxyborohydride (509 mg, 2.4 mmol) was added to the resulting suspension. The reaction mixture was stirred at RT for 1 hr and then poured into ethyl acetate (60 ml). It was washed with saturated sodium bicarbonate (10 ml×2), saturated brine solution (10 ml) and evaporated to give a dark brown residue. The residue was purified by column chromatography, eluting with 3% MeOH in CH₂Cl₂ to give a brown viscous oil which contained a small amount of 4-fluorobenzyl alcohol. The oil was re-chromatographed to give white crystals of the title compound, 337 mg.

MS (chemical ionization): 411 (M+1, 48%), 189 (100%).

¹H NMR (600 MHz, CDCl₃): δ 7.22-7.28 (m, 3H), 7.14 (t, J=6.24 Hz, 1H), 7.02-7.10 (m, 2H), 6.96 (t, J=7.14 Hz, 2H), 6.76 (d, J=6.12 Hz, 1H), 6.64 (m, 2H), 5.50-5.76 (bs, 1H), 5.14 (s, 1H), 3.96 (d, J=12.57 Hz, 1H), 3.06 (d, J=12.57 Hz, 1H), 2.72 (q, J=12.57, 3.35 Hz, 1H), 2.60 (q, J=9.18, 2.94 Hz, 1H), 2.44 (bd, J=5.51 Hz, 2H), 1.98 (t, J=7.34 Hz, 1H), 1.94 (t, J=9.17 Hz, 1H), 1.09 (d, J=10 Hz, 6H).

Calcd for C₂₄H₂₇FN₂OS0.1CH₂Cl₂ 0.25HOCH₂PhF: C, 68.95; H, 6.42; F, 5.27; N, 6.22; S, 7.12. Found: C, 68.92; H, 6.48; F=5.47; N, 6.09; S, 6.82.

### EXAMPLE 12

3-((S)-((2S,5R)-4-(4-chlorobenzyl)-2,5-dimethyl-1-piperazinyl)(3-thienyl)methyl)phenol

A suspension of 3-((S)-((2S,5R)-4-allyl-2,5-dimethyl-1-piperazinyl)(3-thienyl)methyl)phenol (prepared as in Example 3, 3.192 g, 9.32 mmol), chlorotris(triphenylphosphine)rhodium (I) (1.0 g, 1.08 mmol) in 4:1 acetonitrile/water (100 ml) was gently refluxed for 1 hr. Acetonitrile was slowly distilled off while 100 ml of 4:1 acetonitrile/water was added into the reaction mixture in such a rate to maintain the continuous distillation. Volume was reduced to ˜40 mL over 3 hrs. The resulting dark brown suspension was evaporated to a fluffy solid which was purified by column chromatography eluting with 5% and then 10% ammoniated MeOH in CH₂Cl₂ to give 3-((S)-((2S,5R)-2,5-dimethyl-1-piperazinyl)(3-thienyl)methyl)phenol as a dark brown solid, 2.34 g (83% yield).

To a solution of 3-((S)-((2S,5R)-2,5-dimethyl-1-piperazinyl)(3-thienyl)methyl)phenol (363 mg, 1.2 mmol) and 4-chlorobenzaldehyde (253 mg, 1.8 mmol) in anhydrous tetrahydrofuran (8 mL) under nitrogen was added acetic acid (100 mg), followed by sodium triacetoxyborohydride (509 mg, 2.4 mmol) after 30 min. The reaction mixture was stirred at room temperature for 1 hr and then poured into ethyl acetate (60 mL). It was washed with saturated sodium bicarbonate (10 mL×2), saturated brine solution (10 mL) and evaporated to give a dark brown residue. The residue was purified by column chromatography, eluting with 3% MeOH in CH₂Cl₂ to give the title compound as a light fluffy solid, 194 mg. MS (CI): 427 (M+1, 100%), 189 (85%).

¹H NMR (600 MHz, CDCl₃): δ 7.20-7.28 (m, 5H), 7.18 (t, J=7.80 Hz, 1H), 7.08 (bs, 1H), 7.04 (d, J=4.8 Hz, 1H), 6.81 (d, J=7.2 Hz, 1H), 6.68-6.74 (m, 2H), 5.14 (s, 1H), 4.70-5.10 (bs, 1H), 3.91 (d, J=13.2 Hz, 1H), 3.09 (d, J=13.2 Hz, 1H), 2.73 (q, J=11.4, 3 Hz, 1H), 2.60 (q, J=10.8, 2.4 Hz, 1H), 2.47 (bs, 2H), 1.94 (q, J=20.4, 11.4 Hz, 2H), 1.10 (d, J=6 Hz, 3H), 1.06 (d, J=6 Hz, 3H).

Calcd for C₂₄H₂₇ClN₂OS0.05CH₂Cl₂0.4C₄H₈O₂: C, 66.04; H, 6.55; Cl, 8.36; N, 6.01; S, 6.87. Found: C, 66.23; H, 6.36; Cl, 8.66; N, 5.75; S, 6.47.

While the invention has been illustratively described herein with respect to various illustrative aspects, features and embodiments, it will be appreciated that numerous variations, modifications and other embodiments are possible in the practice of the present invention, and the invention therefore is to be broadly construed as encompassing all such variations, modifications and other embodiments, within its spirit and scope.

We claim:

1. A diarylmethylpiperazine compound of the general formula (I): 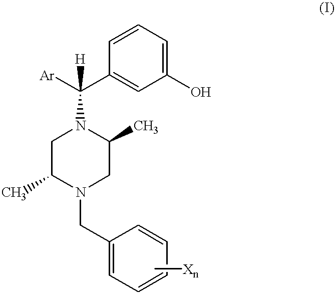
wherein:
Ar is a 5- or 6-member carbocyclic or heterocyclic aromatic ring with atoms selected from the group consisting of carbon, nitrogen, oxygen and sulfur, and having on a first carbon atom thereof a substituent Y and on a second ring carbon thereof a substituent R¹,
Y is selected from the group consisting of:
hydrogen;
halogen;
C₁-C₆ alkyl, C₂-C₆ alkenyl, C₂-C₆ alkynyl;
C₁-C₆ haloalkyl;
C₁-C₆ alkoxy;
C₃-C₆ cycloalkoxy;
sulfides of the formula SR⁸ where R⁸ is C₁-C₆ alkyl, C₂-C₆ alkenyl, C₂-C₆ alkynyl, C₃-C₆ cycloalkyl, arylalkyl having a C₅-C₁₀ aryl moiety and an C₁-C₆ alkyl moiety, or C₅-C₁₀ aryl;
sulfoxides of the formula SOR⁸ where R⁸ is the same as above;
sulfones of the formula SO₂R⁸ where R⁸ is the same as above;
nitrile;
C₁-C₆ acyl;
alkoxycarbonylamino (carbamoyl) of the formula NHCO₂R⁸ where R⁸ is the same as above;
carboxylic acid, or an ester, amide, or salt thereof;
aminomethyl of the formula CH₂NR⁹R¹⁰ where R⁹ and R¹⁰ may be the same or different, and may be hydrogen, C₁-C₆ alkyl, C₂-C₆ alkenyl, C₂-C₆ alkynyl, C₂-C₆ hydroxyalkyl, C₂-C₆ methoxyalkyl, C₃-C₆ cycloalkyl, or C₅-C₁₀ aryl, or R⁹ and R¹⁰ together may form a ring of 5 or 6 atoms, the ring atoms selected from the group consisting of N and C;
carboxamides of the formula CONR⁹R¹⁰ where R⁹ and R¹⁰ are the same as above, or C₂-C₃₀ peptide conjugates thereof; and
sulfonamides of the formula SO₂NR⁹R¹⁰ where R⁹ and R¹⁰ are the same as above;
R¹ is hydrogen, halogen, or C₁-C₄ alkyl, C₂-C₄ alkenyl, C₁-C₄ alkynyl;
X is halo (fluorine, bromine, chlorine, iodine); and
n is from 0 to 5;
or a pharmaceutically acceptable ester or salt thereof.

2. A compound of formula (I) according to claim 1, wherein Ar is selected from the group consisting of thiophenyl, thiazolyl, furanyl, pyrrolyl, phenyl, and pyridyl.

3. A compound of formula (I) according to claim 1, wherein Ar is selected from the group consisting of phenyl and thiophenyl.

4. A compound of formula (I) according to claim 1, wherein Ar is thiophenyl.

5. A compound of formula (II): 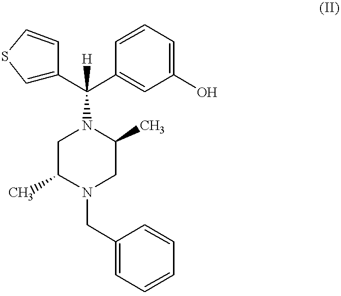
or a pharmaceutically acceptable ester or salt thereof.

6. 3-((S)-((2S,5R)-4-Benzyl-2,5-dimethyl-1-piperazinyl)(3-thienyl)methyl)phenol.

7. A pharmaceutically acceptable ester or salt of 3-((S)-((2S,5R)-4-Benzyl-2,5-dimethyl-1-piperazinyl)(3-thienyl)methyl)phenol.

8. A compound of formula (I) according to claim 1, wherein X is fluoro.

9. A compound of formula (I) according to claim 8, wherein n is 1, 2, 3 or 4.

10. A pharmaceutical composition comprising a diarylmethylpiperazine compound as in claim 1 and a pharmaceutically acceptable carrier therefor.

11. A pharmaceutical composition comprising (a) 3-((S)-((2S,5R)-4-Benzyl-2,5-dimethyl-1-piperazinyl)(3-thienyl)methyl)phenol or a pharmaceutically acceptable ester or salt thereof, and (b) a pharmaceutically acceptable carrier therefor.

12. A diarylmethylbenylpiperazine compound selected from the group consisting of compounds of the following formulae (II) to (XV), and their pharmaceutically acceptable esters and salts: 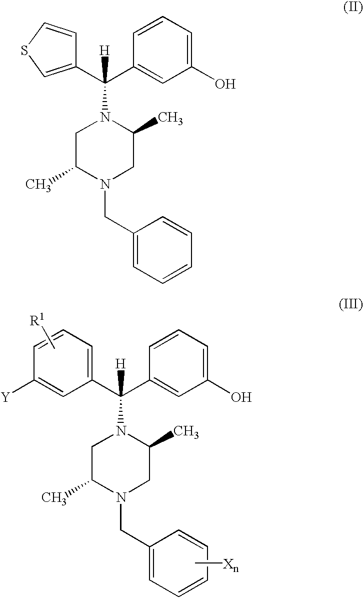
wherein
Y is selected from the group consisting of:
hydrogen;
halogen;
C₁-C₆ alkyl, C₂-C₆ alkenyl, C₂-C₆ alkynyl;
C₁-C₆ haloalkyl;
C₁-C₆ alkoxy;
C₃-C₆ cycloalkoxy;
sulfides of the formula SR⁸ where R⁸ is C₁-C₆ alkyl, C₂-C₆ alkenyl, C₂-C₆ alkynyl, C₃-C₆ cycloalkyl, arylalkyl having a C₅-C₁₀ aryl moiety and an C₁-C₆ alkyl moiety, or C₅-C₁₀ aryl;
sulfoxides of the formula SOR⁸ where R⁸ is the same as above;
sulfones of the formula SO₂R⁸ where R⁸ is the same as above;
nitrile;
C₁-C₆ acyl;
alkoxycarbonylamino (carbamoyl) of the formula NHCO₂R⁸ where R⁸ is the same as above;
carboxylic acid, or an ester, amide, or salt thereof;
aminomethyl of the formula CH₂NR⁹R¹⁰ where R⁹ and R¹⁰ may be the same or different, and may be hydrogen, C₁-C₆ alkyl, C₂-C₆ alkenyl, C₂-C₆ alkynyl, C₂-C₆ hydroxyalkyl, C₂-C₆ methoxyalkyl, C₃-C₆ cycloalkyl, or C₅-C₁₀ aryl, or R⁹ and R¹⁰ together may form a ring of 5 or 6 atoms, the ring atoms selected from the group consisting of N and C; carboxamides of the formula CONR⁹R¹⁰ where R⁹ and R¹⁰ are the same as above, or C₂-C₃₀ peptide conjugates thereof, and
sulfonamides of the formula SO₂NR⁹R¹⁰ where R⁹ and R¹⁰ are the same as above;
R¹ is hydrogen, halogen, or C₁-C₄ alkyl, C₂-C₄ alkenyl, C₁-C₄ alkynyl;
X is halo (fluorine, bromine, chlorine, iodine); and
n is from 0 to 5;

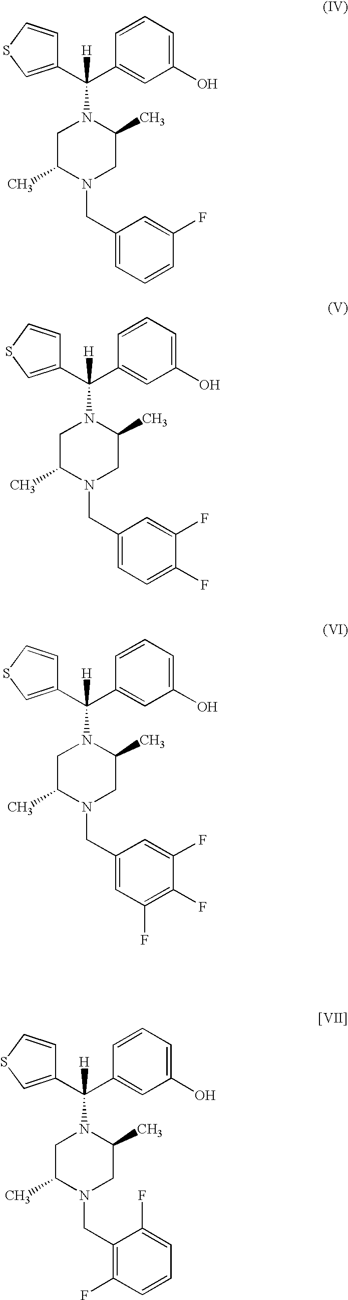

13. A compound of formula (VIII): 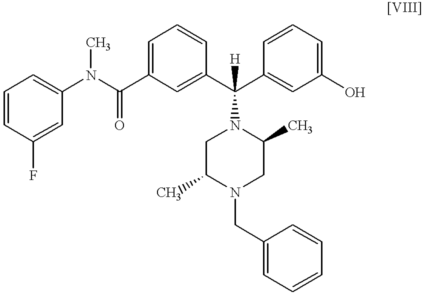
or a pharmaceutically acceptable ester or salt thereof.

14. 3-((R)-((2S,5R)-2,5-dimethyl-4-(4-fluoro-benzyl)-1-piperazinyl)-3-hydroxybenzyl)-N-(3-fluorophenyl)-N-methylbenzamide.

15. A pharmaceutically acceptable ester or salt of 3-((R)-((2S,5R)-2,5-dimethyl-4-(4-fluorobenzyl)-1-piperazinyl)-3-hydroxybenzyl)-N-(3-fluorophenyl)-N-methylbenzamide.

16. A method of treatment or prophylaxis of a disease state or condition selected from the group consisting of drug addiction, alcohol addiction, drug overdose, cough, lung edema, gastrointestinal disorders, arthritis, psoriasis, asthma, inflammatory bowel disease, disorders of respiratory function, functional bowel disease, irritable bowel syndrome, diarrhea, functional distension, pain, non-ulcerogenic dyspepsia, urogenital tract disorders, organ transplant rejection, skin graft rejection, cardiac disorders, mental disorders, cognitive disorders; emesis; respiratory depression; acne; and skin lesions, comprising administering to a subject in need of such treatment or prophylaxis an effective amount therefor of a diarylmethylbenzylpiperazine compound, optionally substituted on an aromatic ring of said benzyl substituent with one or more halo substituents, or a pharmaceutically acceptable ester or salt of such compound.

17. The method of claim 16, wherein the subject is an animal subject.

18. The method of claim 16, wherein the subject is a human subject.

19. The method of claim 16, wherein the diarylmethylbenzylpiperazine compound, optionally substituted on an aromatic ring of said benzyl substituent with one or more halo substituents, or a pharmaceutically acceptable ester or salt of such compound, comprises a diarylmethylpiperazine compound of the general formula (I): 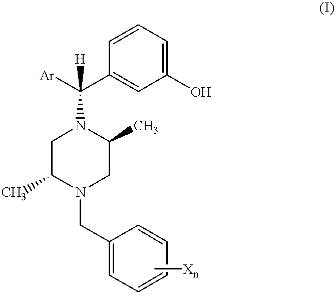
wherein:
Ar is a 5- or 6-member carbocyclic or heterocyclic aromatic ring with atoms selected from the group consisting of carbon, nitrogen, oxygen and sulfur, and having on a first carbon atom thereof a substituent Y and on a second ring carbon thereof a substituent R¹,
Y is selected from the group consisting of:
hydrogen;
halogen;
C₁-C₆ alkyl, C₂-C₆ alkenyl, C₂-C₆ alkynyl;
C₁-C₆ haloalkyl;
C₁-C₆ alkoxy;
C₃-C₆ cycloalkoxy;
sulfides of the formula SR⁸ where R⁸ is C₁-C₆ alkyl, C₂-C₆ alkenyl, C₂-C₆ alkynyl, C₃-C₆ cycloalkyl, arylalkyl having a C₅-C₁₀ aryl moiety and an C₁-C₆ alkyl moiety, or C₅-C₁₀ aryl;
sulfoxides of the formula SOR⁸ where R⁸ is the same as above;
sulfones of the formula SO₂R⁸ where R⁸ is the same as above;
nitrile;
C₁-C₆ acyl;
alkoxycarbonylamino (carbamoyl) of the formula NHCO₂R⁸ where R⁸ is the same as above;
carboxylic acid, or an ester, amide, or salt thereof;
aminomethyl of the formula CH₂NR⁹R¹⁰ where R⁹ and R¹⁰ may be the same or different, and may be hydrogen, C₁-C₆ alkyl, C₂-C₆ alkenyl, C₂-C₆ alkynyl, C₂-C₆ hydroxyalkyl, C₂-C₆ methoxyalkyl, C₃-C₆ cycloalkyl, or C₅-C₁₀ aryl, or R⁹ and R¹⁰ together may form a ring of 5 or 6 atoms, the ring atoms selected from the group consisting of N and C;
carboxamides of the formula CONR⁹R¹⁰ where R⁹ and R¹⁰ are the same as above, or C₂-C₃₀ peptide conjugates thereof; and
sulfonamides of the formula SO₂NR⁹R¹⁰ where R⁹ and R¹⁰ are the same as above;
R¹ is hydrogen, halogen, or C₁-C₄ alkyl, C₂-C₄ alkenyl, C₁-C₄ alkynyl;
X is halo (fluorine, bromine, chlorine, iodine); and
n is from 0 to 5;
or a pharmaceutically acceptable ester or salt thereof.

20. The method of claim 16, wherein the diarylmethylbenzylpiperazine compound, optionally substituted on an aromatic ring of said benzyl substituent with one or more halo substituents, or a pharmaceutically acceptable ester or salt of such compound, comprises a compound selected from the group consisting of compounds of formulae (II)-(XV) and their pharmaceutically acceptable esters and salts.

21. The method of claim 16, wherein the diarylmethylbenzylpiperazine compound, optionally substituted on an aromatic ring of said benzyl substituent with one or more halo substituents, or a pharmaceutically acceptable ester or salt of such compound, comprises a compound of formula (II) or a pharmaceutically acceptable ester or salt thereof.

22. A compound of formula (I) according to claim 1, having an enantiopurity of at least 98%.

23. A compound of formula (I) according to claim 1, having an enantiopurity of at least 99%.

24. 3-((S)-((2S,5R)-4-Benzyl-2,5-dimethyl-1-piperazinyl)(3-thienyl)methyl)phenol or a pharmaceutically acceptable ester or salt thereof, having an enantiopurity of at least 98%.

25. 3-((S)-((2S,5R)-4-Benzyl-2,5-dimethyl-1-piperazinyl)(3-thienyl)methyl)phenol or a pharmaceutically acceptable ester or salt thereof, having an enantiopurity of at least 99%.

26. 3-((R)-((2S,5R)-2,5-dimethyl-4-(4-fluoro-benzyl)-1-piperazinyl)-3-hydroxybenzyl)-N-(3-fluorophenyl)-N-methylbenzamide or a pharmaceutically acceptable ester or salt thereof, having an enantiopurity of at least 98%.

27. 3-((R)-((2S,5R)-2,5-dimethyl-4-(4-fluoro-benzyl)-1-piperazinyl)-3-hydroxybenzyl)-N-(3-fluorophenyl)-N-methylbenzamide or a pharmaceutically acceptable ester or salt thereof, having an enantiopurity of at least 99%.

28. A method of treatment or prophylaxis of a disease state or physiological condition, comprising administering to a subject in need thereof, a diarylmethylpiperazine compound having a safety ratio of at least 12.

29. A method according to claim 28, wherein the disease state or physiological condition is condition selected from the group consisting of drug addiction, alcohol addiction, drug overdose, mental illness, cough, lung edema, gastro-intestinal disorders, arthritis, psoriasis, asthma, inflammatory bowel disease, disorders of respiratory function, functional bowel disease, irritable bowel syndrome, diarrhea, functional distension, pain, non-ulcerogenic dyspepsia, urogenital tract disorders, organ transplant rejection, skin graft rejection, cardiac disorders, mental disorders, cognitive disorders; emesis; respiratory depression; acne and skin lesions.

30. A method according to claim 28, wherein the diarylmethylpiperazine compound comprises a diarylmethylpiperazine compound substituted on the piperazine ring with a benzyl substituent which in turn is optionally substituted on the phenyl ring of the benzyl group with at least one halogen substituent.

31. A method according to claim 28, wherein the safety ratio is at least 15.

32. A method according to claim 28, wherein the diarylmethylpiperazine compound comprises 3-((S)-((2S,5R)-4-Benzyl-2,5-dimethyl-1-piperazinyl)(3-thienyl)methyl)phenol or a pharmaceutically acceptable ester or salt thereof.

33. A method of diagnosis of degeneration or dysfunction of delta opioid receptors associated with a disease state or physiological condition involving tissue or discrete cellular loci comprising said receptors, said method comprising administration of a labeled delta opioid receptor-binding compound to a subject to effect binding of the compound, and determining the extent of binding of the compound to the delta opioid receptors in the subject, as diagnostic information for said diagnosis.

34. A method according to claim 33, wherein said delta opioid receptor-binding compound comprises a diarylmethylpiperazine compound optionally substituted on the piperazine ring with a benzyl substituent which in turn is optionally substituted on the phenyl ring of the benzyl group with at least one halogen substituent.

35. A method according to claim 33, wherein the delta opioid receptor-binding compound is labeled by fluorescent, isotopic or reporter group labeling.

36. A method according to claim 33, wherein said extent of binding of the compound to the delta opioid receptors in the subject, is determined using positron emission tomography in a brain scan of the subject.

37. A method of treatment or prophylaxis of pain, comprising administering to a subject in need of such treatment or prophylaxis an effective amount therefor of 3-((S)-((2S,5R)-4-Benzyl-2,5-dimethyl-1-piperazinyl)(3-thienyl)methyl)phenol or a pharmaceutically acceptable ester or salt thereof.

38. A method of treatment or prophylaxis of pain, comprising administering to a subject in need of such treatment or prophylaxis an effective amount therefor of 3-((R)-((2S,5R)-2,5-dimethyl-4-(4-fluoro-benzyl)-1-piperazinyl)-3-hydroxybenzyl)-N-(3-fluorophenyl)-N-methylbenzamide or a pharmaceutically acceptable ester or salt thereof, having an enantiopurity of at east 98%.

39. A method of inducing analgesia in a subject in need thereof, comprising administering to said subject an analgesically effective amount of a diarylmethylbenzylpiperazine compound, optionally substituted on an aromatic ring of said benzyl substituent with one or more halo substituents, or a pharmaceutically acceptable ester or salt of such compound.

40. A diarylmethylpiperazine compound substituted on the piperazine ring with a benzyl substituent which in turn is optionally substituted on the phenyl ring of the benzyl group with at least one halogen substituent.
